(19) United States
(12) Patent Application Publication
(10) Pub. No.: US 2018/0312924 A1
(43) Pub. Date: Nov. 1, 2018

(54) PROTEIN AND GENE ANALYSIS FROM SAME SAMPLE

(71) Applicants: Michael Joseph Pugia, Ganger, IN (US); Zane Baird, Brigham City, UT (US); Zehui Cao, Carmel, IN (US)

(72) Inventors: Michael Joseph Pugia, Ganger, IN (US); Zane Baird, Brigham City, UT (US); Zehui Cao, Carmel, IN (US)

(21) Appl. No.: 15/949,639

(22) Filed: Apr. 10, 2018

Related U.S. Application Data
(60) Provisional application No. 62/490,088, filed on Apr. 26, 2017.

(51) Int. Cl. C12Q 1/6883; G01N 33/68; G01N 1/40

(52) U.S. Cl. CPC C12Q 1/6883; G01N 2001/4088; G01N 1/4077; G01N 33/6803

(57) ABSTRACT

A method is provided for the collection, purification and analysis of antigens and nucleic acids from the same sample, said method comprising: (a) collecting and purifying said antigens and nucleic acids; and (b) analyzing said antigens by means not destructive to the captured nucleic acids.

This application claims the priority benefit under 35 U.S.C. section 119 of U.S. provisional patent application No. 62/490,088 entitled “Protein And Gene Analysis From Same Sample” filed on Apr. 26, 2017; and which is in its entirety herein incorporated by reference.

### BACKGROUND OF THE INVENTION

The invention relates to methods for selective enrichment and analysis of genes and antigens from the same sample. In some aspects, the invention relates to methods, apparatus and kits for selectively enriching, amplifying and detecting one or more different populations of genes and antigens in a sample suspected of containing one or more different populations of non-rare genes and antigens. In some aspects, the invention relates to methods and kits for detecting one or more different populations of genes and antigens that are freely circulating in samples. In some aspects, the invention relates to methods and kits for detecting one or more different populations of genes and antigens that are associated with cells in a sample suspected of containing one or more different populations of cells.

The current practice for conducting in vitro diagnostic testing of patients involve a combination of POC testing for rapid results (<10 min) on a blood drop (lancing the finger) and sending a blood tube to central laboratory for the results to be returned within 1-3 days. Most tests are not available in the POC as the systems are either insensitive, inaccurate, unable to run all test on a single drop of blood or more often than not, unable to do the complex array of methods needed to provide timely and cost effective answers for all types of testing. Meanwhile, central laboratory tests require a blood draw that must be done by skilled professionals.

This 1-3-day lag time makes patient driven events even more complex to follow between multiple doctors, pharmacies, laboratories and illness events. Meanwhile the pharmacy controls the prescription records across events and doctors and pharmacists are the information source for the patient. Additionally, the sample for one analysis is different than the collection for another. Contamination and stability are an issue too. Better patient characterization leads to better outcomes. But medicine is often too complicated and requires professional personal involvement.

One solution is to conduct all analysis at the patient care site, e.g. point of care testing, to decrease the risk of contamination and loss. However, many analyses require multiple different machines or readers and one system is not possible for all testing, requiring different aliquots of samples to be send to different readers for each type of testing (U.S. Pat. Nos. 8,088,593; 8,158,430; 8,984,932 and 9,128,015). An additional problem is POC testing without a blood draw (mL) requires very small sample volumes (μL) from a drop of blood obtained through a finger stick. The smaller the sample volume, the more ideally the usages. A sample volume of 1 μL is desired to reduce the pain of a finger stick and a sample greater than 30 μL is clearly not desired. This smaller sample volume increases the need for high sensitivity even more due to the reduced number of molecular copies present in a μL sample. While higher concentration testing in the mM to μM range are more possible, it is very difficult to do testing for low concentration testing in nM or less needed for rare nucleic acids and proteins (1 to 100,000 copies). While amplification for nucleic acids for low copy numbers is possible, it is difficult to complete the test in less than 30 min for rare and impure nucleic acids.

Alternatively, sample transport approaches have been used to collect small sample blood volume (<30 μL) at the POC in a specialized transport device which allow a liquid to dilute the sample and prepare for transport. (U.S. Pat. No. 7,291,497). This allows the test to be conducted at centralized locations. In some cases these devices integrate a lancet for generation of the blood drop (WO2002/056751) and in other case stabilized the blood onto a membrane using micron sized capillaries and a membrane to separate the blood (U.S. Pat. No. 4,980,068). While these transport devices remove the need for blood tubes and allow POC collection, the collection devices still suffer from the reduced number of molecular copies present in small samples and require that the proteins and nucleic acids be recovered and removed from the collection device for analysis. Additionally, these methods do not allow simultaneous recovery of rare nucleic acids and proteins (1 to 100,000 copies).

While the simultaneous recovery of rare nucleic acids and proteins from the same sample are known (Moldovan J. Cell. Mol. Med. 2014 18, 3; 371-390, & Tolosa BioTechniques 43:799-804), these methods have issues. Impurities due to other nucleic acids, proteins, enzymes, metabolites, cells and sample components can act as interferences and make analysis insensitive or prone to false results. The reagents and materials used for isolation and purification can equally cause insensitive or false results. The rare nucleic acids and proteins must be simultaneous isolated and concentrated to be separated and stabilized from impurities, therefore reagents used must be compatible with both the rare nucleic acids and proteins. Additionally, cell free rare molecular may need to be separated from cell rare molecule in the same sample. While centrifugal separation of cells from plasma works, this is not a POC collection method (Lowes Int. J. Mol. Sci. 2016, 17, 1505).

Current isolation and purification methods typically require cell lysis with a nonionic detergent to free proteins and nucleic acids from cells, followed by acid phenol chloroform extraction and spin columm separation of RNA onto a glass fiber filter, elution of binding and unbound RNA to separate small RNA from large RNA. Some of the earliest procedures known for isolation of nucleic acids are based on using glass fiber or silica material (Vogelstein B, Gillespie D., Proc Natl Acad Sci 1979:79;615-19). Silica coated magnetic particles have been found useful to isolate, separate and concentrate nucleic acids (WO03/058649, U.S. Pat. No. 8,846,897 and U.S. Pat. No. 8,703,931). Here the beads are separated by a magnetic field and washed to remove proteins, nucleases, and other cellular impurities. The nucleic acids are eluted in a small volume of buffer for subsequent analysis. However these methods are non-selective and not able to capture all molecules, while the rare molecules captured remain extremely impure and contaminated. Other approaches to purify rare molecule are more specific, and use affinity purification by hybridization oilgos or antibodies captured onto magnetic beads (U.S. Pat. No. 5,512,439). The beads are separated by a magnetic field and washed to only leave the rare molecules bound. However even here the rare molecules, whether nucleic acids or proteins, are sensitive to the effect of reagents used in extraction and typically less than 70% of the original captured material survive. Other methods, such as punching, lifting or laser microdissection are time consuming and slow.

The problem is further complicated as some nucleic acids and proteins can be unstable. For example, prokaryotic mRNA only has a 2 min half-life and eukaryotic mRNA has a 30 min to 5 h half-life. While DNA and proteins are relatively stable, the action of enzyme and other chemicals in the sample can alter the DNA and proteins. Integrity problems include degradation, fragmentation, and binding. Therefore methods are sensitive to the effect of timing of blood sampling prior to analysis as a result of degradation of the rare molecules. While fixation, for example with formaldehyde, can stabilize nucleic acids and proteins, fixation causes problems such as fragmentation, cross linking and chemical modification. While fixation can be reversed and minimized to reduce modification, the rare molecules purified from fixed samples are often not good candidates for downstream applications that require full-length molecule such as, for example, polymerase chain reaction methods.

The problem of purity and stability of rare molecules is further exacerbated by the chemicals used in the isolation methods. Methods that employ reagents such as, for example, detergents, solvents or phenols, can damage the rare molecules. Nucleases, protease and inhibitors contamination can reduce amplification of isolated rare molecules.

Filtration is another method used for the separation and washing of cells or particles with rare molecules. Filtration relies on using a porous matrix and an effective method to sort rare cells by size or other nature for pre-enrichment. During filtration smaller non rare cells are lost and larger rare cells isolated. However, as mentioned above filtration techniques can only yield low 0.1% purity or less, thus again highly accurate and sensitive detection methods are required. The current state of the art for rare molecule purification and isolation has several issues which keep it from being applied to all testing and collection environments. The simultaneous collection and purification of nucleic acids and antigen in a stabilized form remain needed, especially from samples which are of small volumes (μL).

### SUMMARY OF THE INVENTION

The invention provides a method for the collection, purification and analysis of antigens and nucleic acids from the same sample, said method comprising: (a) collecting and purifying said antigens and nucleic acids; and (b) analyzing said antigens by means not destructive to the captured nucleic acids.

Some examples in accordance with the principles described herein are directed to collection, purification and analysis of antigens and nucleic acids from the same sample, such that proteins and nucleic acids are retained and antigens are measured in a means not destructive to captured nucleic acids. In some aspects, the invention relates to methods, apparatus and kits for selectively enriching, amplifying and detecting one or more different populations of nucleic acids or antigen as analytes in a sample suspected of containing the analytes among one or more different populations of nucleic acids or antigens. In some aspects, the invention relates to methods and kits for detecting one or more different populations of rare nucleic acids or antigens that are freely circulating in samples. In some aspects, the invention relates to methods and kits for detecting one or more different populations of nucleic acids or antigens associated with rare cells in a sample suspected of containing one or more different populations of rare cells and non-rare cells.

In some embodiments, the antigens and nucleic acids can be retained in or on a cell, particle or droplet. In some embodiments, the antigens and nucleic acids can be captured on the same cell, particle or droplet or on separate cell, particle or droplet. In some embodiments, use of size exclusion filtration is used to retain the antigens and nucleic acids in or on a cell, particle or droplet on to porous matrix. In some embodiments, undesired antigens and nucleic acids are washed away from retained antigens and nucleic acids. In some embodiments, retained antigens and nucleic acids are sealed to be protected from contamination until ready to be analyzed.

In some embodiments, the retained antigens are measured first by releasing an analytical label, which is not destructive to the nucleic acids. In some embodiments, the antigens are measured with an affinity agent and analytical label. In other embodiments, the antigens are retained with an affinity agent. In other embodiments, the nucleic acids are released from the sample after antigens are measured. In some examples, the nucleic acids are amplified after release. In still other examples, nucleic acids are released by lysis of cell. In some embodiments, the antigen measurements are used to decide if nucleic acid amplification or measurement is warranted. In still other embodiments, antigens and nucleic acids measurements are related to the time in a biological subject

### BRIEF DESCRIPTION OF THE DRAWINGS

The drawings provided herein are not to scale and are provided for the purpose of facilitating the understanding of certain examples in accordance with the principles described herein and are provided by way of illustration and not limitation on the scope of the appended claims.

FIG. 1 is a schematic depicting an example of a method in accordance with the principles described herein and shows the method as a work process where the biological sample contains antigen 1 of interest and an antigen 2 that is not of interest and considered contamination, as well as an nucleic acid of interest 3, and an nucleic acid 4 that is not of interest and considered contamination, which are collected on a particle or droplet 5. The particle or droplet 5 is then collected on a porous matrix 7 by size exclusion filtration 6 and treated with an affinity agent with an analytical label 8 such that undesired elements 2 and 4 are removed and the antigen 1 and nucleic acid 3 of interest are retained on the porous matrix with the particle or droplet 5. The analytical labels 8 for measuring the antigen are released and measured while the nucleic acid 9 is amplified and measured.

FIG. 2 is another schematic depicting an example of a method in accordance with the principles described herein and shows the method as a work process where the biological sample contains an antigen of interest outside, on or in a cell 1, and an antigen 2 that is not of interest and considered contamination, as well as an nucleic acid of interest 3 outside, on or in a cell, and an nucleic acid 4 that is not of interest and considered contamination, which are collected in a label particle 5 on or in a cell 6. The particle or cell is then collected on a porous matrix 7 by size exclusion filtration 8 and treated with an affinity agent with an analytical label 9 such that undesired elements 2 and 4 are removed and the antigen 1 and nucleic acid 3 of interest are retained on the porous matrix or droplet 5. The analytical labels 10 for measuring the antigen are released and measured while the nucleic acid 11 is amplified and measured.

FIG. 3 is an additional schematic depicting an example of a method in accordance with the principles described herein and shows the steps for simultaneous protein and nucleic acid analysis. In step 1, the sample is obtained from the original source, in this example a human. In step 2 the antigens and nucleic acids are retained, in this example on a porous matrix. In step 3, the retained antigens are measured in way that does not destroy the nucleic acids, in this example by release of an analytical label. In step 4, the retained antigens and nucleic acids are sealed to be protected from contamination until analyzed and transported to a central location. In step 5, the retained antigens and nucleic acids are measured for in depth data. In step 6, the depth data is combined with other data from data banks and stored by time stamp as a record for original source using data analytics. In step 7, the in depth data on antigens and nucleic acids is combined with other data from data banks, shown by item 8, and time stamp as a record for original source using data analytics, shown by item 7. In step 9, the interpretation of the analytical data is reported back to the original source for the next course of action, in this case the human for a new sample.

### DETAILED DESCRIPTION OF THE INVENTION

Methods, apparatus and kits in accordance with the principles described herein have applications in any situation where detection or isolation of rare molecules and cells is needed. Examples of such applications include, by way of illustration and not limitation, diagnostics, biological reactions, chemical reactions, high through-put screening, cloning, clone generation, artificial cells, regenerative cells, compound libraries, cell library screening, cell culturing, protein engineering and other applications.

Some examples in accordance with the principles described herein are directed to methods of molecular analysis. Other examples in accordance with the principles described herein are directed to methods of isolation, characterization and detection of cells, particles, macromolecules, genes, proteins, biochemicals, organic molecules or other compounds. While other examples use droplet sorting for detection of rare cells and cell free molecules. Other examples in accordance with the principles described herein are directed to methods of selective detection of nucleic acids, proteins, cells and biomarkers

Other examples in accordance with the principles described herein are directed to methods of binding and separation of cells and cellular biological content where cells are isolated on a porous matrix and bound materials retained for analysis. In some cases, the cells are artificial cells, modified cells, natural cells, of any and all types.

Some examples in accordance with the principles described herein are directed to methods of binding and separation of nucleic acid, proteins or other biological molecules on particles where particles are isolated on a porous matrix or by magnetic field and the bound materials retained for analysis.

Some examples in accordance with the principles described herein are directed to methods of detecting one or more different populations of nucleic acids, proteins or other biological rare molecules in a sample suspected of containing the one or more different populations of rare molecules and non-rare molecules. These nucleic acids, proteins or other biological molecules can be used as ligand for measuring cells, enzymes, proteases, receptors, proteins, nucleic acids, peptidases, proteins, inhibitors and the like by acting on formation or binding of said molecules. These molecules can be formed as metabolites, natural or man-made origin, such as biological, therapeutics, or others.

Examples in accordance with the principles described herein are directed to methods and kits for analysis of antigens, nucleic acids or other biological molecules. Other examples in accordance with the principles described herein are directed to apparatus for analysis.

Common terminology used to describe this invention are “droplet”, “affinity agent”, “size exclusion filtration” and “analytical label” which are defined herein below.

A “droplet” is a a compartment to hold nanoliter (nL) volumes of biological fluidics and compounds. The droplet can contain compounds and be considered “full”. The droplet can lack compounds and be considered “empty”. The droplet can be “solid like” inside and comprised of a solid like material inside and considered like a “bead”. The droplet can be “liquid like” inside and comprised of a liquid like material and considered like a “emulsion”.

The term “affinity agent” refers to a molecule capable of selectively binding to a specific molecule or a specific type of molecules. The affinity agent can directly bind the variations of analyte of interest, or be directed to an affinity tag. Affinity agent can be attached to a capture particle or label particles or bind a particle through electrostatic, hydrophobic, spatial, ionic or other interactions attracting the variations of analyte or an affinity tag to the affinity agent.

The term “label particle” refers to a particle bound to analytical label and affinity agents by a linkage. The term “capture particle” refers to a particle attached to an additional affinity agent by a linkage and used to capture the variation of analyte.

“Size exclusion filtration” is the use of a porous matrix to separate cell, particle or droplet. and the contents from the rest of the samples. The contents of the cell, particle or droplet are retained on the porous matrix and are called “retained contents”. “Retained contents” can be antigens and nucleic acids retained in or on cell, particle or droplets. Pore diameters of the porous matrix are kept small enough to retain larger sized cells, particle or droplets and their contents. Size exclusion filtration allow washing to remove unbound material, or material not in or on cell, particle or droplets or associated with retained contents.

The term “analytical label” refers to an optical, mass, or electrochemical label capable of being imaged or detected either directly on the porous matrix or in liquid sample containing the released analytical labels from the particles. Analytical label can be attached to an affinity agent for variations of analyte, or to a label particle. Additionally, the analytical label can be released from an affinity agent, or a label particle by breaking a linkage. The analytical label can be used to identify the affinity agent, particle labels or variations of analyte. The analytical label can be used as an identification code for the affinity agent, label particle or variations of analyte. The analytical label can be a polypeptide, peptide and protein. The analytical label can be measured with an internal standard as a calibrator which is structurally similar or identical to the analytical label.

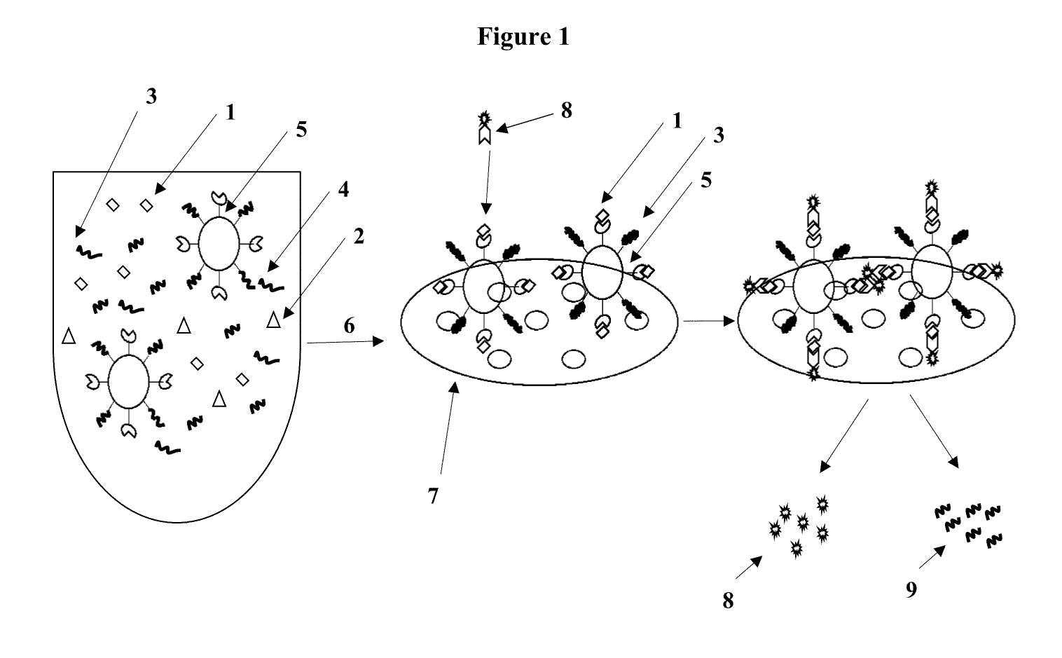

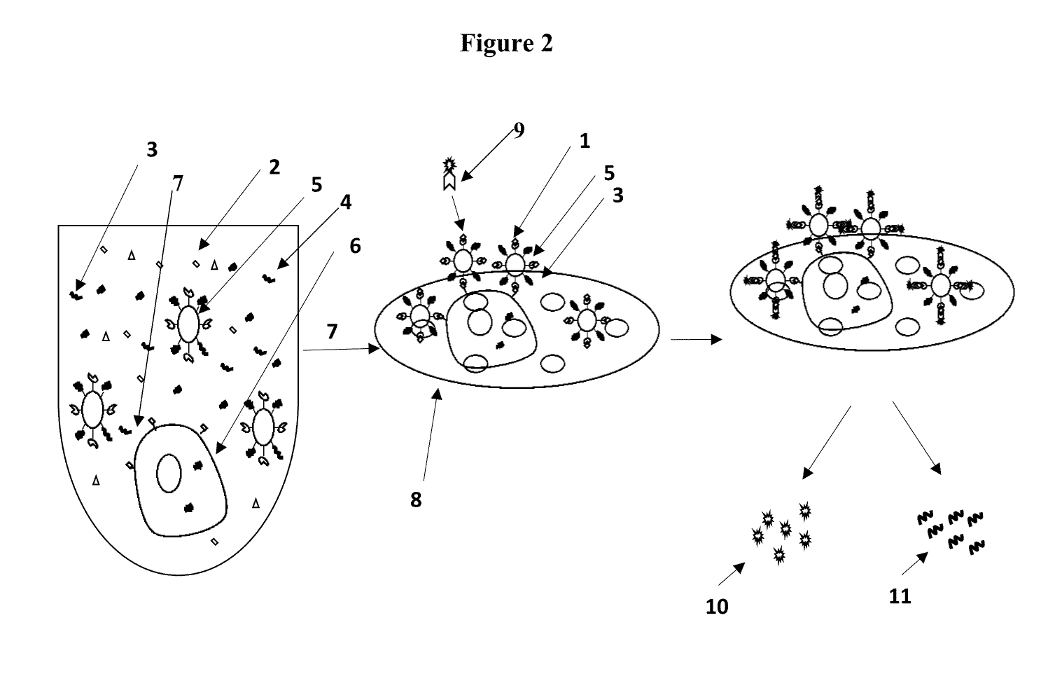

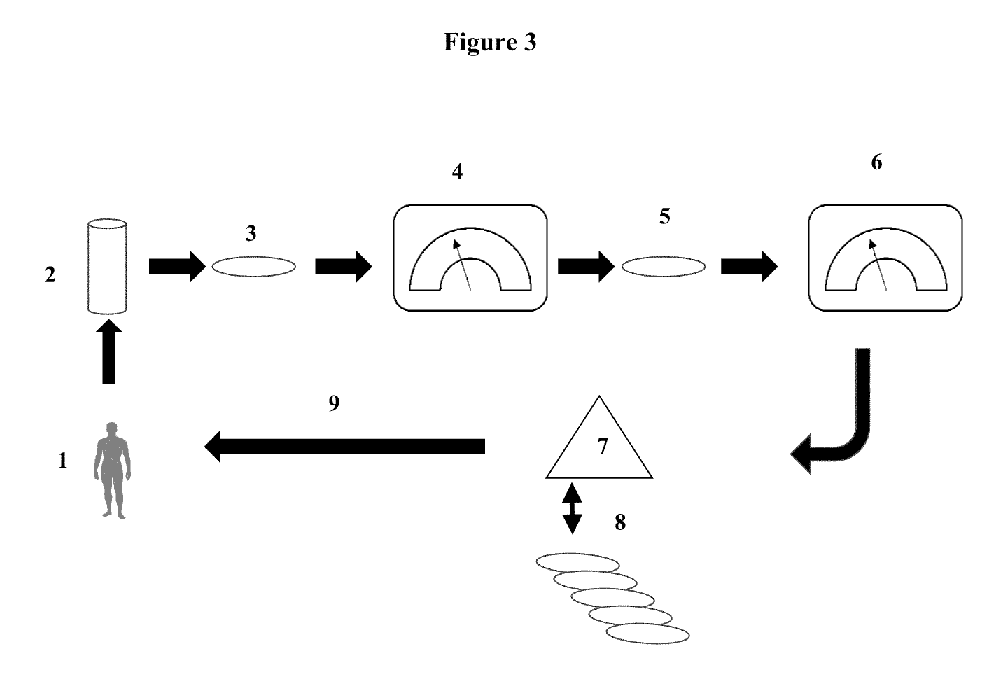

An example of a method for detection of rare molecules in accordance with the principles described herein is depicted in FIGS. 1, 2 and 3. In this examples, antigens and nucleic acids in a sample are captured on or in droplets, particles or cells. Undesired materials are washed way, such that proteins and genes remain captured on particles retained on a porous matrix. A measurement of the antigens is obtained in a means not destructive to captured nucleic acids. The residual sample is sealed and protected from contamination. The retained nucleic acids are amplified for detection.

In other examples, the nucleic acids are amplified and measured after capturing and antigen detection while in other cases, the nucleic acids are amplified and measured after capturing but not before antigen detection. In still other examples, the antigens are measured by label particles with analytical labels that are releasable and non-destructive to genes. In still other examples the nucleic acids and antigens are captured on the same or different cells, particles or droplets retained on a porous matrix.

### Examples of Droplets

A droplet is a micro-sphere defined as a compartment to hold nanoliter (nL)) to microliter (μL) volume of biological fluidics and compounds. The composition of the droplet and biological fluidics can be solvent, organic molecules, matrix, biochemicals, polymers or other macromolecules. The biological fluids can be aqueous or polar and contain solutes, polymers, surfactants, emulsifiers, macromolecules, or particles in addition to other components. The droplet can be “solid like” inside and comprised of a solid like material inside and considered like a “bead”. The droplet can be “liquid like” inside and comprised of a liquid like material and considered like a “emulsion”.

The droplets are made from “emulsion” when it is separated into two immiscible liquids, namely a generally “aqueous phase” held inside the droplet and a generally “oil phase” outside the droplet. Emulsifiers, surfactants, polar, apolar solvents, solutes and the droplets are considered components of an “emulsion”. The stabilization or destabilization of an “emulsion” can lead to continuation of the “emulsion” or separation of aqueous and oil into separate phases. A “droplet” is created when an emulsion is created causing the separation of two immiscible liquids, “aqueous phase” held inside the droplet and a generally “oil phase” outside the droplet. Aqueous phases can include hydrophilic chemical and biochemicals, water, polar protic solvents, polar aprotic solvent and mixtures thereof. Oil phase can include organic solvents, oils such as vegetable, synthetic, animal products, lipids and other lipophilic chemicals and biochemicals. The emulsion can be oil-in-water, water in oil, water in oil in water, and oil in water in oil. Emulsifiers, emulgents, surfactants are components of the emulsion used to change the surface energy of the droplet or the hydrophilic/hydrophobic (lipophilic) balance, and include anionic, cationic, nonionic and amphoteric surfactants, as well as naturally occurring materials. Emulsion instability can be caused by sedimentation, aggregation, coalescence and phase inversion. The emulsion stability can be impacted by oil polarity, temperature, nature of solids in the droplet, droplet size and pH. These properties can be used to stabilize or destabilize droplets and contents.

The droplets can be made from a library of compounds. The “compounds” can be macromolecules, organic molecules, biomolecules, chemicals, nucleic acids, proteins, peptides, antigens, cells, organoids, cells clusters, tissues, capture particles, label particles or others compounds that have unique identities and can be isolated as elements of one or more into liquid droplets (1 μm to 500 μm diameter). The droplet can contain compounds and be considered “full”. The droplet can lack compounds and be considered “empty”. The droplet size can be varied to change the space allowed for a compound, for example the droplet can be varied from 1 to 400 μm in diameter that hold nL to μL volumes. The diameter of liquid droplets can be adjusted for size of compound libraries, for example the particle size, compound size and the likes.

Each droplet can additionally contain affinity agents and labeled particles bound to the antigens and/or nucleic acids. These label particles can serve as identification markers for antigens and nucleic acids. The droplet size can be varied to change the space allowed for a compound, for example the droplet can be nm to μm in diameter. An “excess” of empty droplets to full droplets means a ratio of no greater than 10 full droplets:100 empty droplets such that the ratio of empty to full droplet allows for dilution of sample interference. The number of empty droplets compared to the number of full droplets can be large (>97%) with only (<3%) of droplets created full. In some examples the ratio of full to empty droplets is about 1 to 100, or about 1 to 1000, or about 1 to 10000. “Rapid” droplet generation and sorting means at least >10²/sec.

A “library of compounds” can be a set of “elements” of a common type including organic molecules, biochemicals, genes, particulates, cells, or macromolecules. A “library of compounds” contains any number of unique group members. Generally the library is a group of compounds of similar size and nature and contains some molecular differences between group members. A library of compounds can be a group of “variations of antigens and nucleic acids” or variations of nucleic acids such as sequence differences. The “library of compounds” can be captured onto “capture particles”, macromolecules or cells. The “library of compounds” can be captured through an “affinity agent”. Encapsulation of a compound library in a droplet is typically at least 10² different group members. The term “variations of genes and proteins” is a part, piece, fragment or modification of a polypeptide, protein, and a nucleic acid, whether RNA or DNA, of biological or non-biological origin. Binding and association reactions also lead to additional differences in “variations of peptides and proteins” as well as a variable domain sequences in genes or gene products.

### Examples of Nucleic Acid Amplification and Measurement

The measurement of nucleic acid can be achieved by conventional nucleic acid assays. The nucleic acids can be subjected to one or more amplifications that can take several days for analysis time. Amplification techniques include, but not limited to, enzymatic amplification such as, for example, polymerase chain reaction (PCR), ligase chain reaction (LCR), nucleic acid sequence based amplification (NASBA), Q-β-replicase amplification, 3 SR amplification (specific for RNA and similar to NASBA except that the RNAase-H activity is present in the reverse transcriptase), transcription mediated amplification (TMA) (similar to NASBA in utilizing two enzymes in a self-sustained sequence replication), whole genome amplification (WGA) with or without a secondary amplification such as, e.g., PCR, multiple displacement amplification (MDA) with or without a secondary amplification such as, e.g., PCR, whole transcriptome amplification (WTA) with or without a secondary amplification such as, e.g., PCR or reverse transcriptase PCR.

Droplets can serve as compartments for reactions to produce nucleic acids and nucleic acids with analytical labels. For example, amplification of isolated material, growth of cells, growth of cell cluster, enzymatic reaction, protein synthesis, metabolism and other biochemical reactions. This can increase the copy number of proteins or molecules from artificial cells so they can be directed for detection, characterization and identification. Additionally the reactions can replicate genetic material for additional copies, for example, reverse transcriptase (RT) reactions to convert RNA to DNA, polymerase chain reactions (PCR), and polymerase (Pol) amplification to make more genetic copies for analysis and convert DNA to cDNA. This can increase the copy number of genetic material for detection, sequencing and archival storage. For example, a PCR amplification can be done by adding template to a microwell and allow making of 10⁶ replicates for each copy of template by heating at 95° C. for 5 min, then 20 cycles of heating at 94° C. for 1 min, 60° C. for 1 min, and 72° C. for 1 min. In another example, cell free RNA and DNA can be converted by reverse transcription of RNA to cDNA and Pol amplification of cfDNA to cDNA. Other example includes cDNA amplicon library preparation for sequencing.

### Examples of Variations of Proteins and Genes

In accordance with the principle described, “variations of genes and proteins” can derive from a gene or protein from biological or non-biological origin. The variations of genes and proteins can be used to measure diseases. The variations of genes and proteins can be the result of diseases or intentional reactions. The variations of genes and proteins can result in proteins and peptides of man-made or natural origin and include bioactive and non-bioactive genes or proteins such as those used in medical devices, for therapeutic use, for diagnostic use, used for measurement of processes, and those used as food, in agriculture, in production, as pro or pre biotics, in micro-organism or cellular production, as chemicals for processes, for growth, measurement or control of cells, used for food safety and environmental assessment, used in veterinary products, and used in cosmetics. The fragments and products can be used to measure enzymes, peptidase and other reactions of interest based on formation of variations of genes and proteins. The variations of genes and proteins can be used to measure natural or synthetic inhibition of enzymes, peptidase and other reaction of interest based on lack formation of variations of genes and proteins.

The variations of peptides and proteins can be the result of translation, or post-translational modification by enzymatic or non-enzymatic processes. Post-translational modification refers to the covalent modification of proteins during or after protein biosynthesis. Post-translational modification can be through enzymatic or non-enzymatic chemical reaction. Phosphorylation is a very common mechanism for regulating the activity of enzymes and is the most common post-translational modification. Enzymes can be oxidoreductases, hydrolases, lyases, isomerases, ligases or transferases as known commonly in enzyme taxonomy databases, such as http://enzyme.expasy.org/ and http://www.enzyme-database.org/ which have more than 6000 entries.

Common modification of variations of peptides and proteins include the addition of hydrophobic groups for membrane localization, addition of cofactors for enhanced enzymatic activity, diphthalide formation, hypusine formation, ethanolamine phosphoglycerol attachment, diphthalide formation, acylation, alkylation amide bond formation, amide bond formation such as amino acid addition or amidation, butyrylation gamma-carboxylation dependent on Vitamin K[15], glycosylation, the addition of a glycosyl group to either arginine, asparagine, cysteine, hydroxylysine, serine, threonine, tyrosine, or tryptophan resulting in a glycoprotein, malonylationhydroxylation, iodination, nucleotide addition such as ADP-ribosylation, phosphate ester (O-linked) or phosphoramidate (N-linked) formation such as phosphorylation or adenylylation, propionylation, pyroglutamate formation, S-glutathionylation, S-nitrosylation, and S-sulfenylation (also known as S-sulphenylation, succinylation or sulfation). Non-enzymatic modification include the attachment of sugars, carbamylation, carbonylation or intentional recombination or synthetic conjugation such as biotinylation or addition of affinity tags, such as His oxidation, rormation of disulfide bonds between Cys residues or pegylation.

Common reagents for intentional fragmentation to variations of peptides and proteins include peptidases or reagents known to react with peptides and proteins. Intentional fragmentation can generate specific fragments using predicted cleavage sites for proteases (also termed peptidases or proteinases) and chemicals known to react with peptide and protein sequence. Common peptidases and chemicals for intentional fragmentation include Arg-C, Asp-N, BNPS-skatole NCS/urea, caspase, chymotrypsin (low specificity), Clostripain, CNBr, enterokinase, factor Xa, formic acid, Glu-C, granzyme B, HRV3C protease, hydroxylamine, iodosobenzoic acid, Lys-C, Lys-N, mild acid hydrolysis, NBS, NTCB, elastase, pepsin A, prolyl endopeptidase, proteinase K, TEV protease, thermolysin, thrombin, and trypsin. Common reagents for intentional inhibition of fragmentation include peptidase and chemical inhibitors for peptidases and chemicals listed above.

### Examples of Affinity Agent

An affinity agent is a molecule capable of binding selectively to a rare molecule or an analytical label. Selective binding involves the specific recognition of one molecule or one type of molecules as compared to substantially less recognition of other molecules. The term “binding” or “bound” refers to the manner in which two moieties are associated to one another. An affinity agent can be a immunoglobulin, protein, peptide, metal, carbohydrate, metal chelator, nucleic acid or other molecules capable of binding selectively to a particular rare molecule or an analytical label type.

Examples of nucleic acids include but not limited to natural and made-made oligomeric nucleic acids. The oligomeric nucleic acid may be any polymeric form of nucleotides of any length, either deoxyribonucleotides or ribonucleotides, or analogs thereof. The following are non-limiting examples of polynucleotides: coding or non-coding regions of a gene or gene fragment, loci (locus) defined from linkage analysis, exons, introns, messenger RNA (mRNA), transfer RNA, ribosomal RNA, ribozymes, cDNA, interfering RNA (siRNA), microRNA (miRNA), peptide nucleic acids (PNA), locked nucleic acids (LNA), xeno nucleic acids (XNA), recombinant polynucleotides, branched polynucleotides, plasmids, vectors, isolated DNA of any sequence, isolated RNA of any sequence, nucleic acid probes, and primers. A polynucleotide may comprise modified nucleotides, such as methylated nucleotides and nucleotide analogs, and/or modified backbone, such as that in PNA and that in phosphorothioate polynucleotides. If present, modifications to the polynucleotide structure may be imparted before or after assembly of the polymer.

The sequence of polynucleotides may be interrupted by non-nucleotide components. A polynucleotide may be further modified, such as by conjugation with a labeling component. The terms “isolated nucleic acid” and “isolated polynucleotide” are used interchangeably; a nucleic acid or polynucleotide is considered “isolated” if it: (1) is not associated with all or a portion of a polynucleotide in which the “isolated polynucleotide” is found in nature, (2) is linked to a polynucleotide to which it is not linked in nature, or (3) does not occur in nature as part of a longer sequence.

The affinity agents which are immunoglobulins may include complete antibodies or fragments thereof. Immunoglobulins include all of the various classes and isotypes, such as IgA, IgD, IgE, IgG1, IgG2a, IgG2b and IgG3, IgM, etc. Fragments thereof may include Fab, Fv, F(ab′)2, and Fab′, for example. In addition, aggregates, polymers, and conjugates of immunoglobulins or their fragments can be used where appropriate so long as binding affinity for a particular molecule is maintained. Antibodies can be monoclonal or polyclonal. Such antibodies can be prepared by techniques that are well known in the art such as immunization of a host and collection of sera (polyclonal), or by preparing continuous hybrid cell lines and collecting the secreted protein (monoclonal), or by cloning and expressing nucleotide sequences or mutagenized versions thereof coding at least for the amino acid sequences of natural antibodies required for specific binding.

Polyclonal antibodies and monoclonal antibodies may be prepared by techniques that are well known in the art. For example, in one approach monoclonal antibodies are obtained by somatic cell hybridization techniques. Monoclonal antibodies may be produced according to the standard techniques of Köhler and Milstein (Nature 265:495-497, 1975). Reviews of monoclonal antibody techniques are found in Lymphocyte Hybridomas, ed. Melchers, et al. Springer-Verlag (New York 1978), Nature 266: 495 (1977), Science 208: 692 (1980), and Methods of Enzymology 73 (Part B): 3-46 (1981). In general, monoclonal antibodies can be purified by known techniques such as, but not limited to, chromatography, e.g., DEAE chromatography, ABx chromatography, and HPLC chromatography, and filtration.

An affinity agent can additionally be a “cell affinity agent” capable of binding selectively to a rare molecule which is used for typing a rare cell or measuring a intracellular process of a cell. These rare cell markers can be immunoglobulins that specifically recognize and bind to antigens associated with a particular cell type and whereby the antigens are components of the cell. The cell affinity agent is capable of being absorbed into or onto the cell. The term “cell affinity agent” refers to a rare cell typing marker capable of binding selectively to the rare cell. Selective cell binding typically involves binding between molecules that is relatively dependent on specific structures of binding pair. Selective binding does not rely on non-specific recognition.

### Examples of Analytical Labels

In some examples in accordance with the principles described herein, analytical labels are employed for detection and measurement of different populations of variation of analyte in the methods, kits and apparatus. Analytical labels are molecules, metals, charges, ions, atoms or electrons that are detectable using analytical methods to yield information about the presence and amounts of variation of analyte over other molecules in the sample. The principles described herein are directed to methods using analytical labels of detecting one or more different populations of variation of analyte in a sample suspected of containing the one or more different populations of rare molecules and non-rare molecules. In some examples, the variations of analyte are in a cell or from a cell. In other examples, the variations of analyte are free of cells or “cell free” assays. In other examples, the variation of analyte are cells which are rare or “rare cell assay”. In some examples in accordance with the principles described herein, the one or more different populations of variation of analyte is retained on the porous matrix or capture particles, and reacted to generate and release an analytical label from the porous matrix or capture particles.

The analytical labels can be detected when retained on the porous matrix and released from the membrane into analysis liquid. The analytical labels can be detected when retained on the capture particle or cell and released from the capture particle or cell into analysis liquid. In some examples, the analytical labels are released from analytical label precursor into the analysis liquid without the variation of analyte. In other examples, the analytical labels are released from analytical label precursor into the analysis liquid with the variation of analyte. In other examples, the analytical labels are not released from analytical label precursor into the analysis liquid with the variation of analyte.

The porous matrix or analysis liquid can be subjected to analysis to determine the presence and/or amount of each different analytical labels. The presence and/or amount of each different analytical label are related to the presence and/or amount of each different population of target rare molecules in the sample. The analytical labels can be measured by optical, electrochemical, or mass spectrographic methods as optical analytical labels, electrochemical analytical labels or mass spectrometry analytical labels. Optional presence and/or amount of each different types of labels whether optical analytical labels, electrochemical analytical labels or mass spectrometry analytical labels can be related to each other to determine the presence and/or amount of each different population of target rare molecules retained on the porous substrate or capture particles, or released into the analysis liquid.

In some examples, the analysis liquid with analytical labels can go in to a liquid receiving area that is sampled by an analyzer. In other examples, the analysis liquid with analytical labels can be retained on the porous matrix that is sampled by an analyzer. In other case, the liquid receiving area can be inside an analyzer and the analysis liquid with analytical labels can go directly into an analyzer. In some analysis examples, the porous matrix is removed and places in analyzer either on top and/or bottom and placed in an analyzer or reader where analytical labels are analyzed and converted to information about the presence and/or amount of each different rare targets.

In some in accordance with the principles described herein, analytical labels are released from analytical label precursor. In many examples, analytical labels can be generated after reaction with a chemical to break a bond. In other examples, analytical labels are generated from analytical label precursor substrate which are derivatives that undergo reaction with an enzyme such as horseradish peroxidase, alkaline phosphatase, β-galactosidase, flavo-oxidase enzyme, urease or methyltransferase to name a few, to release the label. In other examples, the analytical labels can be generated after reaction with an electron or ion, such as an electro-chemiluminescence (ECL) label.

As mentioned above, one or more linking groups X—Y are a moiety that is cleavable by a cleavage agent. The nature of the cleavage agent is dependent on the nature of the cleavable moiety. Cleavage of the cleavable moiety may be achieved by chemical or physical methods, involving one or more of oxidation, reduction, solvolysis, e.g., hydrolysis, photolysis, thermolysis, electrolysis, sonication, and chemical substitution, for example. Examples of cleavable moieties and corresponding cleavage agents, by way of illustration and not limitation, include disulfide that may be cleaved using a reducing agent, e.g., a thiol; diols that may be cleaved using an oxidation agent, e.g., periodate; diketones that may be cleaved by permanganate or osmium tetroxide; ether, esters, diazo linkages or oxime linkages that may be cleaved with hydrosulfite; β-sulfones, which may be cleaved under basic conditions; tetralkylammonium, trialkylsulfonium, tetralkylphosphonium, where the α-carbon is activated, e.g., with carbonyl or nitro, that may be cleaved with base; ester and thioester linkages that may be cleaved using a hydrolysis agent such as, e.g., hydroxylamine, ammonia or trialkylamine (e.g., trimethylamine or triethylamine) under alkaline conditions; quinones where elimination occurs with reduction; substituted benzyl ethers that can be cleaved photolytically; carbonates that can be cleaved thermally; metal chelates where the ligands can be displaced with a higher affinity ligand; thioethers that may be cleaved with singlet oxygen; hydrazone linkages that are cleavable under acidic conditions; quaternary ammonium salts (cleavable by, e.g., aqueous sodium hydroxide); trifluoroacetic acid-cleavable moieties such as, e.g., benzyl alcohol derivatives, teicoplanin aglycone, acetals and thioacetals; thioethers that may be cleaved using, e.g., HF or cresol; sulfonyls (cleavable by, e.g., trifluoromethane sulfonic acid, trifluoroacetic acid, or thioanisole); nucleophile-cleavable sites such as phthalamide (cleavable, e.g., with substituted hydrazines); ionic association (attraction of oppositely charged moieties) where cleavage may be realized by changing the ionic strength of the medium, adding a disruptive ionic substance, lowering or raising the pH, adding a surfactant, sonication, and adding charged chemicals; and photocleavable bonds that are cleavable with light having an appropriate wavelength such as, e.g., UV light at 300 nm or greater; for example.

In one example, a cleavable linkage may be formed using conjugation with N-succinimidyl 3-(2-pyridyldithio)propionate) (SPDP), which comprises a disulfide bond. For example, a label particle comprising an amine functionality is conjugated to SPDP and the resulting conjugate can then be reacted with an analytical label comprising a thiol functionality, which results in the linkage of the mass label moiety to the conjugate. A disulfide reducing agent (such as, for example, dithiothreitol (DTT) or tris(2-carboxyethyl)phosphine (TCEP)) may be employed as an alteration agent to release a thiolated peptide as an analytical label.

The phrase “optical analytical labels” refers to a group of molecules having illumination with light of a particular wavelength, such as: a chemiluminescent label like luminol, isoluminol, acridinium esters, adamantyl 1, 2-dioxetane aryl phosphate, metals derivatives of or others commonly available to researchers in the field; a fluorescent label like fluorescein, lanthanide metals, Hoechst 33258, R-phycocyanin, B-phycoerythrin, R-phycoerythrin, rhodamine, DyLight Dyes™, Texas red, fluorescent proteins, quantum dots, metals or other list commonly available to researchers in the field (see http://www.fluorophores.org/) or; a chromogenic label tetramethylbenzidine (TMB), particles, metals or others. Optical analytical labels are detectable by optical methods like microscope, camera, optical reader, colorimeter, fluorometer, luminometer, reflectrometer, and others.

The phrase “electrochemical analytical labels” refers to potentiometric, capacitive and redox active compounds such as: metals like Pt, Ag, Pd, Au and many others; or particles like gold sols, graphene oxides and many others; or electron transport molecules like ferrocene, ferrocyanide, Os(VI)bipy and many others; or electrochemical redox active molecules like aromatic alcohols and amines such as 4-aminophenyl phosphate, 2-naphthol, para-nitrophenol phosphate; thiols or disulfides such as those on aromatics, aliphatics, amino acids, peptides and proteins; aromatic heterocyclic containing non-carbon ring atoms, like, oxygen, nitrogen, or sulfur, such as imidazoles, indoles, quinolones, thiazole, benzofuran and many others. Electrochemical analytical labels are detectable by impedance, capacitance, amperometry, electrochemical impedance spectroscopy and other measurement.

A label particle can include 1 to about 10⁸ analytical labels, or about 10 to about 10⁴ analytical labels, or about 10³ to about 10⁵ analytical labels, or about 10⁴ to about 10⁸ analytical labels, or about 10⁶ to about 10⁸ analytical labels, for example. The label particle can comprise proteins, polypeptides, polymers, particles, carbohydrates, nucleic acids, lipids or other macromolecules capable of including multiple repeating units of analytical labels by attachment through the X-Y linkage. Multiple label particles allow amplification as every label particles can generate many analytical labels.

The phrase “mass label” or “mass labels” refers to a group of molecules having unique masses below 3 kDA such that each unique mass corresponds to, and is used to determine the presence and/or amount of, each different population of target rare molecules. The mass labels are molecules of defined mass and include, but are not limited to, polypeptides, polymers, fatty acids, carbohydrates, organic amines, nucleic acids, and organic alcohols, for example, whose mass can be varied by substitution and changing size, for example. In the case of polymeric materials, the number of repeating units is adjusted such that the mass is in a region that does not overlap with a background mass from the sample. The mass label generates a unique mass pattern due to structure and fragmentation upon ionization.

The “mass label” is any molecule that results in a unique mass. The mass label bound to the label particle may through the action of the alteration agent be converted to another mass label by cleavage, by reaction with a moiety, by derivatization, or by addition or by subtraction of molecules, charges or atoms, for example, or a combination of two or more of the above.

### Examples of Label and Capture Particle

Affinity agent can be attached to analytical labels and/or particles for purpose of detection or isolation of rare molecules. This attachment can occur through “label particles” which are in turn attached to mass labels. Affinity agents can also be attached to “capture particles” which allow separation of bound and unbound analytical labels or rare molecule. This attachment to capture and label can be prepared by directly attaching the affinity agent in a “linking group”. The terms “attached” or “attachment” refers to the manner in which two moieties are connected by a direct bond between the two moieties or a linking group between the two moieties. This allows the method to be multiplexed for more than one result at a time. Alternatively, affinity agent can be attached to analytical labels and/or mass label using additional “binding partners”. The phrase “binding partner” refers to a molecule that is a member of a specific binding pair of affinity agent and “affinity tags” that bind each other and not the analytical labels or rare molecules. In some cases, the affinity agent may be members of an immunological pair such as antigen to antibody or hapten to antibody, biotin to avidin, IgG to protein A, secondary antibody to primary antibody, antibodies to fluorescent labels and other examples of binding pairs.

The “label particle” is a particulate material which can be attached to the affinity agent through a direct linker arm or a binding pair. The “label particle” is also capable of forming X-Y cleavable linkage between label particle and mass label. The size of the label particle is large enough to accommodate one or more mass labels and affinity agents. The ratio of affinity agents or mass label to a single label particle may be 10⁷ to 1, 10⁶ to 1, or 10⁵ to 1, or 10⁴ to 1, or 10³ to 1, or 10² to 1, or 10 to 1, for example. The number of affinity agents and analytical labels associated with the label particle is dependent on one or more of the following, the nature and size of the affinity agent, the nature and size of the label particle, the nature of the linker arm, the number and type of functional groups on the label particle, and the number and type of functional groups on the mass label, for example.

The composition of the label or capture particle entity may be organic or inorganic, magnetic or non-magnetic as a nanoparticle or a micro particle. Organic polymers include, by way of illustration and not limitation, nitrocellulose, cellulose acetate, poly(vinyl chloride), polyacrylamide, polyacrylate, polyethylene, polypropylene, poly(4-methylbutene), polystyrene, poly(methyl methacrylate), poly(hydroxyethyl methacrylate), poly(styrene/divinylbenzene), poly(styrene/acrylate), poly(ethylene terephthalate), dendrimer, melamine resin, nylon, poly(vinyl butyrate), for example, either used by themselves or in conjunction with other materials including latex, microparticle and nanoparticle forms thereof. The particles may also comprise carbon (e.g., carbon nanotubes), metal (e.g., gold, silver, and iron, including metal oxides thereof), colloids, dendrimers, dendrons, and liposomes. In some examples, the label particle may be a silica nanoparticle. In other examples, label particles can be magnetic that have free carboxylic acid, amine or tosyl groups. In other examples, label particles can be mesoporous and include analytical labels inside the label particles.

The diameter of the label or capture particle is dependent on one or more of the following, the nature of the rare molecule, the nature of the sample, the permeability of the cell, the size of the cell, the size of the nucleic acid, the size of the affinity agent, the magnetic forces applied for separation, the nature and the pore size of a filtration matrix, the adhesion of the particle to matrix, the surface of the particle, the surface of the matrix, the liquid ionic strength, liquid surface tension and components in the liquid, and the number, size, shape and molecular structure of associated label particles, for example.

The term “permeability” means the ability of a particles and molecule to enter a cell through the cell wall. In the case of detection of a rare molecule inside the cell, the diameter of the label particles must be small enough to allow the affinity agents to enter the cell. The label particle maybe coated with materials to increase “permeability” like collagenase, peptides, proteins, lipid, surfactants, and other chemicals known to increase particle inclusion into the cell. When a porous matrix is employed in filtration separation step, the diameter of the label particles must be large enough to not pass through the pores of a porous matrix to retain the bound rare molecule on the matrix. In some examples in accordance with the principles described herein, the average diameter of the label particles should be at least about 0.01 microns (10 nm) and not more than about 10 microns In some examples, the particles have an average diameter from about about 0.02 microns to about 0.06 microns, or about 0.03 microns to about 0.1 microns, or about 0.06 microns to about 0.2 microns, or about 0.2 microns to about 1 micron, or about 1 micron to about 3 microns, or about 3 micron to about 10 microns. In some examples, the adhesion of the particles to the surface is so strong that the particle diameter can be smaller than the pore size of the matrix.

The affinity agent can be prepared by directly attaching the affinity agent to carrier or capture particles by linking groups. The linking group between the label particle and the affinity agent may be aliphatic or aromatic bond. The linking groups may comprise a cleavable or non-cleavable linking moiety. Cleavage of the cleavable moiety can be achieved by the same electrochemical reduction used for the mass label but also may be achieved by chemical or physical methods, including oxidation, reduction, solvolysis, e.g. hydrolysis, photolysis, thermolysis, electrolysis, sonication, and chemical substitution, for example. Photocleavable bonds that are cleavable with light having an appropriate wavelength such as, e.g., UV light at 300 nm or greater; for example. The nature of the cleavage agent is dependent on the nature of the cleavable moiety. When heteroatoms are present, oxygen will normally be present as oxy or oxo, bonded to carbon, sulfur, nitrogen or phosphorous; sulfur will be present as thioether or thiono; nitrogen will normally be present as nitro, nitroso or amino, normally bonded to carbon, oxygen, sulfur or phosphorous; phosphorous will be bonded to carbon, sulfur, oxygen or nitrogen, usually as phosphonate and phosphate mono- or diester. Functionalities present in the linking group may include esters, thioesters, amides, thioamides, ethers, ureas, thioureas, guanidines, azo groups, thioethers, carboxylate and so forth. The linking group may also be a macro-molecule such as polysaccharides, peptides, proteins, nucleotides, and dendrimers.

The linking group between the particle and the affinity agent may be a chain of from 1 to about 60 or more atoms, or from 1 to about 50 atoms, or from 1 to about 40 atoms, or from 1 to 30 atoms, or from about 1 to about 20 atoms, or from about 1 to about 10 atoms, each independently selected from the group normally consisting of carbon, oxygen, sulfur, nitrogen, and phosphorous, usually carbon and oxygen. The number of heteroatoms in the linking group may range from about 0 to about 8, from about 1 to about 6, or about 2 to about 4. The atoms of the linking group may be substituted with atoms other than hydrogen, such as, for example, one or more of carbon, oxygen and nitrogen in the form of, e.g., alkyl, aryl, aralkyl, hydroxyl, alkoxy, aryloxy, or aralkoxy groups. As a general rule, the length of a particular linking group can be selected arbitrarily to provide for convenience of synthesis with the proviso that there is minimal interference caused by the linking group with the ability of the linked molecules to perform their function related to the methods disclosed herein.

Obtaining reproducibility in amounts of particle captured after separation and isolation is important for rare molecular analysis. Additionally, knowing the amounts of particles captured that enter a rare cell is important to maximize the amount of specific binding. Knowing the amount of particles remained after washing is important to minimize the amount of non-selective binding. In order to make these determinations, it is helpful that the particles can contain fluorescent, optical or chemiluminescence labels, so that the label particles, can be measured by fluorescence or chemiluminescence by virtue of the presence of a fluorescent or chemiluminescent molecule. The fluorescent and optical molecule can then be measured by microscopic analysis and compared to expected results for samples containing or lacking the analyte. Fluorescent molecule include but not limited to Dylight™, FITC, rhodamine compounds, fluorescent proteins, quantum dots, phycoerythrin, phycocyanin, allophycocyanin, o phthalaldehyde, fluorescent rare earth chelates, amino-coumarins, umbelliferones, oxazines, Texas red, acridones, perylenes, indacines such as, e.g., 4,4-difluoro-4-bora-3a,4a-diaza-s-indacene and variants thereof, 9,10-bis-phenylethynylanthracene, squaraine dyes and fluorescamine, for example. A fluorescent microscope or fluorescent spectrometer may then be used to determine the location and amount of the label particles. Chemiluminescence labels examples include luminol, acridinium esters and acridinium sulfonamides to name a few. Optical labels examples include color particles, gold particles, enzymatic colorimetric reactions to name a few.

### Examples of Porous Matrix and Filtration

Porous matrices are used in “size exclusion filtration” to allow washing away unbound material or material not in full droplets or associated with retained contents. The contents of the droplets are retained on the porous matrix and are called “retained contents”. “Retained contents” can be cells or particles and molecules associated. Full droplets can also be retained with contents on the porous matrix. Pore diameters of the porous matrix are kept small enough to retain larger sized droplets and their contents. “Size exclusion filtration” allow washing away unbound material or material not in full droplets or associated with retained contents.

A porous matrix can be at bottom of micowells to hold the droplets and retained contents on cells and particles. Well diameters must be greater than droplets, cell or particles used to retain the content in a well while still not obstructing washing and allowing washing away undesired materials. Droplet diameter can vary from 1 to 400 μm. Particles can vary from 15 nm to 10 μm in diameter and serve as capture or detection particles. Particles can be associated with other particles or cells. Isolation of the detection particles and cells or capture particles can be used for the detection of rare molecules. Porous matrix are used where the detection particles are sufficiently smaller than the pore size of the matrix such that physically the particles can fall through the pores if not captured. In other examples, the capture particles are sufficiently larger than the pore size of the matrix such that physically the particles cannot fall through the pores. Cells size can variy from 1 μm to 50 μm in diameter. Cells can also be in clusters or spheroids of multiple cells of up to an average diameter of 200 μm. The well diameter is at least 2 times greater than the diameter of droplet, cells, cell clusters or spheroids. This allows individual droplets, cells, cell clusters or spheroids in a well. The ratio of the diameter of the droplet or cells compared to the diameter of the well being less then 10 improves separation to allow sorting one droplet or cells per well.

In some examples, multiple porous matrices can be used to separate cells, particles and droplets into populations of different sizes. A porous matrix with larger pore size is used for filtration before porous matrix with smaller pore size is used, so that cells, particles and droplets larger than the pore size of the first matrix are retained on the first porous matrix while cells, particles and droplets with sizes between the pore size of the first matrix and the pore size of the second matrix are retained on the second porous matrix. When needed, more than 2 porous matrices can be used to separate cells, particles and droplets into even more fractions each with defined size range.

In some methods in accordance with the principles described herein, the sample is incubated with an affinity agent comprised of a mass label and label particle, for each different population of rare molecules. The affinity agent can comprise a specific binding partner that is specific for and binds to one of the populations of the rare molecules, where the rare molecules can be cell bound or cell free. The affinity agent with mass label and label particle is retained on the surface of a membrane of a filtration.

The separation can occur in some examples when a porous matrix employed in filtration separation step is such that the pore diameter is smaller than the diameter of the cell with the rare molecule but larger than the unbound label particles to allow the affinity agents to achieve the benefits of rare molecule capture in accordance with the principles described herein but small enough to pass through the pores of a porous matrix or a porous matrix if they did not capture any rare molecules. In other methods, the porous matrix employed in a filtration separation step is such that the pore diameter is smaller than the diameter of the affinity agents on label particle capable of binding rare molecule but larger than the unbound molecules passing through, which allows the affinity agents to achieve the benefits of rare molecule capture. In still other methods, the affinity agents on label particle can be additionally bound through “binding partners” or a sandwich format to other capture particles, like magnetic particles, or to a surface, like a membrane. In the later case, the capture particles are retained on the surface of the porous membranes.

In all examples, the concentration of the one or more different populations of rare molecules is enhanced over that of the non-rare molecules to form a concentrated sample. In some examples, the sample is subjected to a filtration procedure using a porous matrix that retains the rare molecules while allowing the non-rare molecules to pass through the porous matrix thereby enhancing the concentration of the rare molecules. In the event that one or more rare molecules are non-cellular, i.e., not associated with a cell or other biological particles, the sample is combined with one or more capture particle entities wherein each capture particle entity comprises a binding partner for the non-cellular rare molecule of each of the populations of non-cellular rare molecules to render the non-cellular rare molecules in particulate form, i.e., to form particle-bound non-cellular rare molecules. The combination of the sample and the capture particle entities is held for a period of time and at a temperature to permit the binding of non-cellular rare molecules with corresponding binding partners of the capture particle entities.

Vacuum is applied to the sample on the porous matrix to facilitate passage of non-rare cells and other particles through the matrix. The level of vacuum applied is dependent on one or more of the following, the nature and size of the different populations of rare cells and/or particle reagents, the nature of the porous matrix, and the size of the pores of the porous matrix, for example.

Contact of the sample with the porous matrix is continued for a period of time sufficient to achieve retention of cellular rare molecules and/or particle-bound non-cellular rare molecules on the surface of the porous matrix to obtain a surface of the porous matrix having different populations of rare cells and/or particle-bound rare molecules as discussed above. The period of time is dependent on one or more of the following, the nature and size of the different populations of rare cells and/or particle-bound rare molecules, the nature of the porous matrix, the size of the pores of the porous matrix, the level of vacuum applied to the blood sample on the porous matrix, the volume to be filtered, and the surface area of the porous matrix, for example. In some examples, the period of contact is about 1 minute to about 1 hour, or about 5 minutes to about 1 hour, or about 5 minutes to about 45 minutes, or about 5 minutes to about 30 minutes, or about 5 minutes to about 20 minutes, or about 5 minutes to about 10 minutes, or about 10 minutes to about 1 hour, or about 10 minutes to about 45 minutes, or about 10 minutes to about 30 minutes, or about 10 minutes to about 20 minutes.

The amount of each different affinity agent that is employed in the methods in accordance with the principles described herein is dependent on one or more of the following, the nature and potential amount of each different population of rare molecules, the nature of the mass label, the nature of attachment, the nature of the affinity agent, the nature of a cell if present, the nature of a particle if employed, and the amount and nature of a blocking agent if employed, for example. In some examples, the amount of each different modified affinity agent employed is about 0.001 μg/μL to about 100 μg/μL, for example.

The porous matrix is a solid material, which is impermeable to liquid (except through one or more pores of the matrix in accordance with the principles described herein. The porous matrix is associated with a porous matrix holder and a liquid holding well. The association between porous matrix and holder can be done with an adhesive. The association between porous matrix in the holder and the liquid holding well can be through direct contact or with a flexible gasket surface.

The porous matrix is a solid or semi-solid material and may be comprised of an organic or inorganic, water insoluble material. The porous matrix is non-bibulous, which means that the membrane is incapable of absorbing liquid. In some examples, the amount of liquid absorbed by the porous matrix is less than about 2% (by volume), or less than about 1%, or less than about 0.5%, or less than about 0.1%, or less than about 0.01%, or 0%. The porous matrix is non-fibrous, which means that the membrane is at least 95% free of fibers, or at least 99% free of fibers, or at least 99.5%, or at least 99.9% free of fibers, or 100% free of fibers.

The porous matrix can have any of a number of shapes such as, for example, track-etched, or planar or flat surface (e.g., strip, disk, film, matrix, and plate). The matrix may be fabricated from a wide variety of materials, which may be naturally occurring or synthetic, polymeric or non-polymeric. The shape of the porous matrix is dependent on one or more of the following, the nature or shape of the holder for the membrane, the nature or shape of the microfluidic surface, the nature or shape of the liquid holding well, the nature or shape of cover surface, for example. In some examples the shape of the porous matrix is circular, oval, rectangular, square, track-etched, planar or flat surface (e.g., strip, disk, film, membrane, and plate).

The porous matrix and holder may be fabricated from a wide variety of materials, which may be naturally occurring or synthetic, polymeric or non-polymeric. Examples, by way of illustration and not limitation, of such materials for fabricating a porous matrix include plastics such as, for example, polycarbonate, poly(vinyl chloride), polyacrylamide, polyacrylate, polyethylene, polypropylene, poly-(4methylbutene), polystyrene, polymethacrylate, poly(ethylene terephthalate), nylon, poly(vinyl butyrate), poly(chlorotrifluoroethylene), poly(vinyl butyrate), polyimide, polyurethane, and parylene; silanes; silicon; silicon nitride; graphite; ceramic material (such, e.g., as alumina, zirconia, PZT, silicon carbide, aluminum nitride); metallic material (such as, e.g., gold, tantalum, tungsten, platinum, and aluminum); glass (such as, e.g., borosilicate, soda lime glass, and PYREX®); and bioresorbable polymers (such as, e.g., polylactic acid, polycaprolactone and polyglycoic acid); for example, either used by themselves or in conjunction with one another and/or with other materials. The materials for fabrication of the porous matrix and holder are non-bibulous and do not include fibrous materials such as cellulose (including paper), nitrocellulose, cellulose acetate, rayon, diacetate, lignins, mineral fibers, fibrous proteins, collagens, synthetic fibers (such as nylons, dacron, olefin, acrylic, polyester fibers, for example) or, other fibrous materials (glass fiber, metallic fibers), which are bibulous and/or permeable and, thus, are not in accordance with the principles described herein. The materials for fabrication of the porous matrix and holder respectively may be the same or different.

The porous matrix for each liquid holding well comprises at least one pore and no more than about 2,000,000 pores per square centimeter (cm²). In some examples, the number of pores of the porous matrix per cm² is 1 to about 2,000,000, for example. The density of pores in the porous matrix is about 1% to about 20%, for example, of the surface area of the porous matrix. In some examples, the size of the pores of a porous matrix is sufficient to preferentially retain liquid while allowing the passage of liquid droplets formed in accordance with the principles described herein. The size of the pores of the porous matrix is dependent on the nature of the liquid, the size of the cell, the size of the capture particle, the size of mass label, the size of an analyte, the size of label particles, the size of non-rare molecules, and the size of non-rare cells, for example. In some examples the average size of the pores of the porous matrixes is about 0.1 to about 100 microns.

Pores within the matrix may be fabricated in accordance with the principles described herein by, for example, microelectromechanical (MEMS) technology, metal oxide semi-conductor (CMOS) technology, micro-manufacturing processes for producing microsieves, laser technology, irradiation, molding, and micromachining, for example, or a combination thereof.

The porous matrix is permanently attached to a holder which can be associated to the bottom of the liquid holding well and to the top of the vacuum manifold where the porous matrix is positioned such that liquid can flow from the liquid holding well to the vacuum manifold. In some examples, the porous matrix in the holder can be associated with a microfluidic surface, and a top or bottom cover surface. The holder may be constructed of any suitable material that is compatible with the material of the porous matrix. Examples of such materials include, by way of example and not limitation, any of the materials listed above for the porous matrix. The material for the housing and for the porous matrix may be the same or may be different. The holder may also be constructed of non-porous glass or plastic film.

Examples of plastic film materials include polystyrene, polyalkylene, polyolefins, epoxies, Teflon®, PET, chloro-fluoroethylenes, polyvinylidene fluoride, PE-TFE, PE-CTFE, liquid crystal polymers, Mylar®, polyester, polymethylpentene, polyphenylene sulfide, and PVC plastic films. The plastic film can be metallized such as with aluminum. The plastic films can have relative low moisture transmission rate, e.g. 0.001 mg per m²-day. The porous matrix may be permanently attached to a holder by adhesion using thermal bonding, mechanical fastening or through use of permanent adhesives such as drying adhesive like polyvinyl acetate, pressure-sensitive adhesives like acrylate-based polymers, contact adhesives like natural rubber and polychloroprene, hot melt adhesives like ethylene-vinyl acetates, and reactive adhesives like polyester, polyol, acrylic, epoxies, polyimides, silicones rubber-based and modified acrylate and polyurethane compositions, natural adhesive like dextrin, casein, lignin. The plastic film or the adhesive can be electrically conductive materials and the conductive material coatings or materials can be patterned across specific regions of the hold surface.

The porous matrix in the holder is generally part of a filtration module where the porous matrix is part of an assembly for convenient use during filtration. The holder does not contain pores and has a surface that is in contact with associated surfaces but is not permanently attached to these surfaces and can be removed. A top gasket may be applied to the removable holder between the liquid holding wells. A bottom gasket may be applied to the removable holder between the manifold for vacuum. A gasket is a flexible material that facilities complete contact upon compression. The holder may be constructed of the gasket material. Examples of gasket shapes include a flat, embossed, patterned, or molded sheets, rings, circles, ovals, with cutout areas to allow sample to flow from porous matrix to vacuum maniford. Examples of gasket materials include paper, rubber, silicone, metal, cork, felt, neoprene, nitrile rubber, fiberglass, polytetrafluoroethylene like PTFE or Teflon or a plastic polymer like polychlorotrifluoroethylene.

In some examples, vacuum is applied to the concentrated and treated sample on the porous matrix to facilitate passage of non-rare cells through the matrix. The level of vacuum applied is dependent on one or more of the following, the nature and size of the different populations of biological particles, the nature of the porous matrix, and the size of the pores of the porous matrix, for example. In some examples, the level of vacuum applied is about 1 millibar to about 200 millibar for example. In some examples the vacuum is an oscillating vacuum, which means that the vacuum is applied intermittently at regular or irregular intervals, which may be, for example, about 1 second to about 600 seconds. In some approaches, vacuum is oscillated from about 0 millibar to about 100 millibar, for example, during some or all of the application of vacuum to the blood sample. Oscillating vacuum is achieved using an on-off switch, for example, and may be conducted automatically or manually.

Contact of the treated sample with the porous matrix is continued for a period of time sufficient to achieve retention of the rare cells or the particle-bound rare molecules on a surface of the porous matrix to obtain a surface of the porous matrix having different populations of rare cells or the particle-bound rare molecules as discussed above. The period of time is dependent on one or more of the following, the nature and size of the different populations of rare cells or particle-bound rare molecules, the nature of the porous matrix, the size of the pores of the porous matrix, the level of vacuum applied to the sample on the porous matrix, the volume to be filtered, and the surface area of the porous matrix, for example. In some examples, the period of contact is about 1 minute to about 2 hours.

### Examples of Rare Molecules

The phrase “rare molecules” refers to a molecule that may be detected in a sample where the rare molecule is indicative of a particular population of molecules. The phrase “population of molecules” refers to a group of rare molecules that share common rare molecules that is specific for the group of rare molecules. The phrase “specific for” means that the common rare molecules distinguish the group of rare molecules from other molecules.

The methods described herein involve trace analysis, i.e., minute amounts of material on the order of 1 to about 100,000 copies of rare cells or rare molecules. Since this process involves trace analysis at the detection limits of the nucleic acid analyzers, these minute amounts of material can only be detected when detection volumes are extremely low, for example, 10-15 liter, so that the concentrations are within the detection. Given associated error is unlikely that “all” of the rare molecules undergo amplification, i.e., converting the minute amounts of material to the order of about 10⁵ to about 10¹⁰ copies for every original rare molecule. The phrase “substantially all” means that at least about 70 to about 99% measured by the reproducibility of the amount of a rare molecule produced.

The phrase “cell free rare molecules” refers to rare molecules that are not bound to a cell and/or that freely circulate in a sample. Such non-cellular rare molecules include biomolecules useful in medical diagnosis and treatments of diseases. Medical diagnosis of diseases includes, but is not limited to, biomarkers for detection of cancer, cardiac damage, cardiovascular disease, neurological disease, hemostasis/hemastasis, fetal maternal assessment, fertility, bone status, hormone levels, vitamins, allergies, autoimmune diseases, hypertension, kidney disease, metabolic disease, diabetes, liver diseases, infectious diseases and other biomolecules useful in medical diagnosis of diseases, for example.

The following are non-limiting examples of samples that rare molecules can be measured in. The sample to be analyzed is one that is suspected of containing rare molecules. The samples may be biological samples or non-biological samples. Biological samples may be from a plant, animal, protists or other living organism including Animalia, fungi, plantae, chromista, or protozoa or other eukaryote species or bacteria, archaea, or other prokaryote species. Non-biological samples include aqueous solutions, environmental, products, chemical reaction production, waste streams, foods, feed stocks, fertilizers, fuels, and the like. Biological samples include biological fluids such as whole blood, serum, plasma, sputum, lymphatic fluid, semen, vaginal mucus, feces, urine, spinal fluid, saliva, stool, cerebral spinal fluid, tears, mucus, or tissues for example. Biological tissue includes, by way of illustration, hair, skin, sections or excised tissues from organs or other body parts, for example. Rare molecules may be from tissues, for example, lung, bronchus, colon, rectum, extra cellular matrix, dermal, vascular, stem, lead, root, seed, flower, pancreas, prostate, breast, liver, bile duct, bladder, ovary, brain, central nervous system, kidney, pelvis, uterine corpus, oral cavity or pharynx or cancers. In many instances, the sample is aqueous such as a urine, whole blood, plasma or serum sample. In other instances the sample must be made into a solution or suspension for testing.

The sample can be one that contains cells such as, for example, non-rare cells and rare cells where rare molecules are detected from the rare cells. The cells containing rare molecules may be from any organism such as, but not limited to, pathogens such as bacteria, virus, fungus, and protozoa; malignant cells such as malignant neoplasms or cancer cells; circulating endothelial cells; circulating tumor cells; circulating cancer stem cells; circulating cancer mesochymal cells; circulating epithelial cells; fetal cells; immune cells (B cells, T cells, macrophages, NK cells, monocytes); and stem cells, for example. In other examples of methods in accordance with the principles described herein, the sample to be tested is a blood sample from a, organism such as, but not limited to, a plant or animal subject. In some examples of methods in accordance with the principles described herein, the sample to be tested is a sample from an organism such as, but not limited to, a mammal subject, for example. Cells with rare molecules may be from a tissue of mammal, for example, lung, bronchus, colon, rectum, pancreas, prostate, breast, liver, bile duct, bladder, ovary, brain, central nervous system, kidney, pelvis, uterine corpus, oral cavity or pharynx, or cancers.

Rare molecule fragments can be used to measure peptidases of interest including those in the MEROPS, which is an on-line database for peptidases (also known as proteases) containing a total of ˜902212 different sequences of aspartic, cysteine, glutamic, metallo, asparagine, serine, threonine and general peptidases catalytics types, which are further categorized and include those listed for the following pathways: 2-Oxocarboxylic acid metabolism, ABC transporters, African trypanosomiasis, Alanine, aspartate and glutamate metabolism, Allograft rejection, Alzheimer's disease, Amino sugar and nucleotide sugar metabolism, Amoebiasis, AMPK signaling pathway, Amyotrophic lateral sclerosis (ALS), Antigen processing and presentation, Apoptosis, Arachidonic acid metabolism, Arginine and proline metabolism, Arrhythmogenic right ventricular cardiomyopathy (ARVC), Asthma, Autoimmune thyroid disease, B cell receptor signaling pathway, Bacterial secretion system, Basal transcription factors, beta-Alanine metabolism, Bile secretion, Biosynthesis of amino acids, Biosynthesis of secondary metabolites, Biosynthesis of unsaturated fatty acids, Biotin metabolism, Bisphenol degradation, Bladder cancer, cAMP signaling pathway, Carbon metabolism, Cardiac muscle contraction, Cell adhesion molecules (CAMs), Cell cycle, Cell cycle—yeast, Chagas disease (American trypanosomiasis), Chemical carcinogenesis, Cholinergic synapse, Colorectal cancer, Complement and coagulation cascades, Cyanoamino acid metabolism, Cysteine and methionine metabolism, Cytokine-cytokine receptor interaction, Cytosolic DNA-sensing pathway, Degradation of aromatic compounds, Dilated cardiomyopathy, Dioxin degradation, DNA replication, Dorso-ventral axis formation, Drug metabolism—other enzymes, Endocrine and other factor-regulated calcium reabsorption, Endocytosis, Epithelial cell signaling in Helicobacter pylori infection, Epstein-Barr virus infection, Estrogen signaling pathway, Fanconi anemia pathway, Fatty acid elongation, Focal adhesion, Folate biosynthesis, FoxO signaling pathway, Glutathione metabolism, Glycerolipid metabolism, Glycerophospholipid metabolism, Glycosyl-phosphatidylinositol(GPI)-anchor biosynthesis, Glyoxylate and dicarboxylate metabolism, GnRH signaling pathway, Graft-versus-host disease, Hedgehog signaling pathway, Hematopoietic cell lineage, Hepatitis B, Herpes simplex infection, HIF-1 signaling pathway, Hippo signaling pathway, Histidine metabolism, Homologous recombination, HTLV-I infection, Huntington's disease, Hypertrophic cardiomyopathy (HCM), Influenza A, Insulin signaling pathway, Legionellosis, Leishmaniasis, Leukocyte transendothelial migration, Lysine biosynthesis, Lysosome, Malaria, MAPK signaling pathway, Meiosis—yeast, Melanoma, Metabolic pathways, Metabolism of xenobiotics by cytochrome P450, Microbial metabolism in diverse environments, MicroRNAs in cancer, Mineral absorption, Mismatch repair, Natural killer cell mediated cytotoxicity, Neuroactive ligand-receptor interaction, NF-kappa B signaling pathway, Nitrogen metabolism, NOD-like receptor signaling pathway, Non-alcoholic fatty liver disease (NAFLD), Notch signaling pathway, Olfactory transduction, Oocyte meiosis, Osteoclast differentiation, Other glycan degradation, Ovarian steroidogenesis, Oxidative phosphorylation, p53 signaling pathway, Pancreatic secretion, Pantothenate and CoA biosynthesis, Parkinson's disease, Pathways in cancer, Penicillin and cephalosporin biosynthesis, Peptidoglycan biosynthesis, Peroxisome, Pertussis, Phagosome, Phenylalanine metabolism, Phenylalanine, tyrosine and tryptophan biosynthesis, Phenylpropanoid biosynthesis, PI3K-Akt signaling pathway, Plant-pathogen interaction, Platelet activation, PPAR signaling pathway, Prion diseases, Proteasome, Protein digestion and absorption, Protein export, Protein processing in endoplasmic reticulum, Proteoglycans in cancer, Purine metabolism, Pyrimidine metabolism, Pyruvate metabolism, Rap1 signaling pathway, Ras signaling pathway, Regulation of actin cytoskeleton, Regulation of autophagy, Renal cell carcinoma, Renin-angiotensin system, Retrograde endocannabinoid signaling, Rheumatoid arthritis, RIG-I-like receptor signalling pathway, RNA degradation, RNA transport, Salivary secretion, Salmonella infection, Serotonergic synapse, Small cell lung cancer, Spliceosome, Staphylococcus aureus infection, Systemic lupus erythematosus, T cell receptor signaling pathway, Taurine and hypotaurine metabolism, Terpenoid backbone biosynthesis, TGF-beta signaling pathway, TNF signaling pathway, Toll-like receptor signaling pathway, Toxoplasmosis, Transcriptional misregulation in cancer, Tryptophan metabolism, Tuberculosis, Two-component system, Type I diabetes mellitus, Ubiquinone and other terpenoid-quinone biosynthesis, Ubiquitin mediated proteolysis, Vancomycin resistance, Viral carcinogenesis, Viral myocarditis, Vitamin digestion and absorption Wnt signaling pathway.

Rare molecule fragments can be used to measure peptidase inhibitor of interest including those in the MEROPS, which is an on-line database for peptidase inhibitors and includes a total of ˜133535 different sequences of different peptidase inhibitor families where a family is a set of homologous peptidase inhibitors with a homology. The homology is shown by a significant similarity in amino acid sequence either to the type inhibitor of the family, or to another protein that has already been shown to be homologous to the type inhibitor, and thus a member of. The reference organism for the family includes ovomucoid inhibitor unit 3 (Meleagris gallopavo), aprotinin (Bos taurus), soybean Kunitz trypsin inhibitor (Glycine max), proteinase inhibitor B (Sagittaria sagittifolia), alpha-1-peptidase inhibitor (Homo sapiens), ascidian trypsin inhibitor (Halocynthia roretzi), ragi seed trypsin/alpha-amylase inhibitor (Eleusine coracana), trypsin inhibitor MCTI-1 (Momordica charantia), Bombyx subtilisin inhibitor (Bombyx mori),peptidase B inhibitor (Saccharomyces cerevisiae), marinostatin (Alteromonas sp.), ecotin (Escherichia coli), Bowman-Birk inhibitor unit 1 (Glycine max), eglin c (Hirudo medicinalis), hirudin (Hirudo medicinalis), antistasin inhibitor unit 1 (Haementeria officinalis), streptomyces subtilisin inhibitor (Streptomyces albogriseolus), secretory leukocyte peptidase inhibitor domain 2 (Homo sapiens), mustard trypsin inhibitor-2 (Sinapis alba), peptidase inhibitor LMPI inhibitor unit 1 (Locusta migratoria), potato peptidase inhibitor II inhibitor unit 1 (Solanum tuberosum), secretogranin V (Homo sapiens), BsuPI peptidase inhibitor (Bacillus subtilis), pinA Lon peptidase inhibitor (Enterobacteria phage T4), cystatin A (Homo sapiens), ovocystatin (Gallus gallus), metallopeptidase inhibitor (Bothrops jararaca), calpastatin inhibitor unit 1 (Homo sapiens), cytotoxic T-lymphocyte antigen-2 alpha (Mus musculus), equistatin inhibitor unit 1 (Actinia equina), survivin (Homo sapiens), aspin (Ascaris suum), saccharopepsin inhibitor (Saccharomyces cerevisiae), timp-1 (Homo sapiens), Streptomyces metallopeptidase inhibitor (Streptomyces nigrescens), potato metallocarboxypeptidase inhibitor (Solanum tuberosum), metallopeptidase inhibitor (Dickeya chrysanthemi), alpha-2-macroglobulin (Homo sapiens), chagasin (Leishmania major), oprin (Didelphis marsupialis), metallocarboxypeptidase A inhibitor (Ascaris suum), leech metallocarboxypeptidase inhibitor (Hirudo medicinalis), latexin (Homo sapiens), clitocypin (Lepista nebularis), proSAAS (Homo sapiens), baculovirus P35 caspase inhibitor (Spodoptera litura nucleopolyhedrovirus), p35 homologue (Amsacta moorei entomopoxvirus), serine carboxypeptidase Y inhibitor (Saccharomyces cerevisiae), tick anticoagulant peptide (Ornithodoros moubata), madanin 1 (Haemaphysalis longicornis), squash aspartic peptidase inhibitor (Cucumis sativus), staphostatin B (Staphylococcus aureus), staphostatin A (Staphylococcus aureus), triabin (Triatoma pallidipennis), pro-eosinophil major basic protein (Homo sapiens), thrombostasin (Haematobia irritans), Lentinus peptidase inhibitor (Lentinula edodes), bromein (Ananas comosus), tick carboxypeptidase inhibitor (Rhipicephalus bursa), streptopain inhibitor (Streptococcus pyogenes), falstatin (Plasmodium falciparum), chimadanin (Haemaphysalis longicornis), (Veronica) trypsin inhibitor (Veronica hederifolia), variegin (Amblyomma variegatum), bacteriophage lambda CM protein (bacteriophage lambda), thrombin inhibitor (Glossina morsitans), anophelin (Anopheles albimanus), Aspergillus elastase inhibitor (Aspergillus fumigatus), AVR2 protein (Passalora fulva), IseA protein (Bacillus subtilis), toxostatin-1 (Toxoplasma gondii), AmFPI-1 (Antheraea mylitta), cvSI-2 (Crassostrea virginica), macrocypin 1 (Macrolepiota procera), HflC (Escherichia coli), oryctin (Oryctes rhinoceros), trypsin inhibitor (Mirabilis jalapa), F1L protein (Vaccinia virus), NvCI carboxypeptidase inhibitor (Nerita versicolor), Sizzled protein (Xenopus laevis), EAPH2 protein (Staphylococcus aureus), and Bowman-Birk-like trypsin inhibitor (Odorrana versabilis). Rare molecule fragments can be used to measure synthetic inhibition of peptidase inhibitor. The aforementioned database also includes thousands of different small molecule inhibitors that can mimic the inhibitory properties for any member of the above listed families.

Rare molecules of metabolic interest include but are not limited to those that impact the concentration of ACC Acetyl Coenzyme A Carboxylase, Adpn Adiponectin, AdipoR Adiponectin Receptor, AG Anhydroglucitol, AGE Advance glycation end products, Akt Protein kinase B, AMBK pre-alpha-1-microglobulin/bikunin, AMPK 5′-AMP activated protein kinase, ASP Acylation stimulating protein, Bik Bikunin, BNP B-type natriuretic peptide, CCL Chemokine (C—C motif) ligand, CINC Cytokine-induced neutrophil chemoattractant, CTF C-Terminal Fragment of Adiponectin Receptor, CRP C-reactive protein, DGAT Acyl CoA diacylglycerol transferase, DPP-IV Dipeptidyl peptidase-IV, EGF Epidermal growth factor, eNOS Endothelial NOS, EPO Erythropoietin, ET Endothelin, Erk Extracellular signal-regulated kinase, FABP Fatty acid-binding protein, FGF Fibroblast growth factor, FFA Free fatty acids, FXR Farnesoid X receptor a, GDF Growth differentiation factor, GH Growth hormone, GIP Glucose-dependent insulinotropic polypeptide, GLP Glucagon-like peptide-1, GSH Glutathione, GHSR Growth hormone secretagogue receptor, GULT Glucose transporters, GCD59 glycated CD59 (aka glyCD59), HbA1c Hemogloblin A1c, HDL High-density lipoprotein, HGF Hepatocyte growth factor, HIF Hypoxia-inducible factor, HMG 3-Hydroxy-3-methylglutaryl CoA reductase, I-α-I Inter-α-inhibitor, Ig-CTF Immunoglobulin attached C-Terminal Fragment of AdipoR, insulin, IDE Insulin-degrading enzyme, IGF Insulin-like growth factor, IGFBP IGF binding proteins, IL Interleukin cytokines, ICAM Intercellular adhesion molecule, JAK STAT Janus kinase/signal transducer and activator of transcription, JNK c-Jun N-terminal kinases, KIM Kidney injury molecule, LCN-2 Lipocalin,LDL Low-density lipoprotein, L-FABP Liver type fatty acid binding protein, LPS Lipopolysaccharide, Lp-PLA2 Lipoprotein-associated phospholipase A2, LXR Liver X receptors, LYVE Endothelial hyaluronan receptor, MAPK Mitogen-activated protein kinase, MCP Monocyte chemotactic protein, MDA Malondialdehyde, MIC Macrophage inhibitory cytokine, MIP Macrophage infammatory protein, MMP Matrix metalloproteinase, MPO Myeloperoxidase, mTOR Mammalian of rapamycin, NADH Nicotinamide adenine dinucleotide, NGF Nerve growth factor, NFκB Nuclear factor kappa-light-chain-enhancer of activated B cells, NGAL Neutrophil gelatinase lipocalin, NOS Nitric oxide synthase NOX NADPH oxidase NPY Neuropeptide Y glucose, insulin, proinsulin, c peptide OHdG Hydroxydeoxyguanosine, oxLDL Oxidized low density lipoprotein, P-α-I pre-interleukin-α-inhibitor, PAI-1 Plasminogen activator inhibitor, PAR Protease-activated receptors, PDF Placental growth factor, PDGF Platelet-derived growth factor, PKA Protein kinase A, PKC Protein kinase C, PI3K Phosphatidylinositol 3-kinase, PLA2 Phosphatidylinositol 3-kinase, PLC Phospholipase C, PPAR Peroxisome proliferator-activated receptor, PPG Postprandial glucose, PS Phosphatidylserine, PR Protienase, PYY Neuropeptide like peptide Y, RAGE Receptors for AGE, ROS Reactive oxygen species, 5100 Calgranulin, sCr Serum creatinine, SGLT2 Sodium-glucose transporter 2, SFRP4 secreted frizzled-related protein 4 precursor, SREBP Sterol regulatory element binding proteins, SMAD Sterile alpha motif domain-containing protein, SOD Superoxide dismutase's TNFR Soluble TNF α receptor, TACE TNFα alpha cleavage protease, TFPI Tissue factor pathway inhibitor, TG Triglycerides, TGF β Transforming growth factor-β, TIMP Tissue inhibitor of metalloproteinases, TNF α Tumor necrosis factors-α, TNFR TNF α receptor, THP Tamm-Horsfall protein, TLR Toll-like receptors, TnI Troponin I, tPA Tissue plasminogen activator, TSP Thrombospondin, Uri Uristatin, uTi Urinary trypsin inhibitor, uPA Urokinase-type plasminogen activator, uPAR uPA receptor, VCAM Vascular cell adhesion molecule, VEGF Vascular endothelial growth factor, and YKL-40 Chitinase-3-like protein.

Rare molecules of interest that are highly expressed by pancreas or found in the pancreas include insulin, proinsulin, c-peptide, PNLIPRP1 pancreatic lipase-related protein 1, SYCN syncollin, PRSS1 protease, serine protease 1, (trypsin 1) Intracellular, CTRB2 chymotrypsinogen B2 Intracellular, CELA2A chymotrypsin-like elastase family, member 2A, CTRB1 chymotrypsinogen B1 Intracellular, CELA3A chymotrypsin-like elastase family, member 3A Intracellular, CELA3B chymotrypsin-like elastase family, member 3B Intracellular, CTRC chymotrypsin C (caldecrin), CPA1 carboxypeptidase A1 (pancreatic) Intracellular, PNLIP pancreatic lipase, and CPB1 carboxypeptidase B1 (tissue), AMY2A amylase, alpha 2A (pancreatic), PDX1 insulin promoter factor 1, MAFA Maf family of transcription factors, GLUT2 Glucose Transporter Type 2, ST8SIA1 Alpha-N-acetylneuraminide alpha-2,8-sialyltransferase, CD9 tetraspanin, ALDH1A3 aldehyde dehydrogenase, CTFR cystic fibrosis transmembrane conductance regulator as well as diabetic auto immune antibodies such as those against GAD, IA-2, IAA, ZnT8 or the like.

Rare molecule fragments include those of insulin, pro-insulin or c peptide generated by the following peptidases known to naturally act on insulin; archaelysin, duodenase, calpain-1, ammodytase subfamily M12B peptidases, ALE1 peptidase, CDF peptidase, cathepsin E, meprin alpha subunit, jerdohagin (Trimeresurus jerdonii), carboxypeptidase E, dibasic processing endopeptidase, yapsin-1, yapsin A, PCSK1 peptidase, aminopeptidase B, PCSK1 peptidase, PCSK2 peptidase, insulysin, matrix metallopeptidase-9 and others. These fragments include but are not limited to the following sequences of SEQ ID NO:1 malwmrllpllallalwgp, SEQ ID NO:2 malwmrllpl, SEQ ID NO:3 allalwgpd, SEQ ID NO:4 aaafvnqhlcgshlvealylvcgergffytpktr, SEQ ID NO:5 paaafvnqhlcgshlvealylvc, SEQ ID NO:6 paaafvnqhlcgs, SEQ ID NO:7 cgshlvealylv, SEQ ID NO:8 vealylvc, SEQ ID NO:9 lvcgergf, SEQ ID NO:10 ffytpk, SEQ ID NO:11 reaedlqvgqvelgggpgagslqplalegsl SEQ ID NO:12 reaedlqvgqve SEQ ID NO:13 lgggpgag SEQ ID NO:14 slqplalegsl SEQ ID NO:15 giveqcctsicslyqlenycn SEQ ID NO:16 giveqcctsicsly SEQ ID NO:17 qlenycn, and SEQ ID NO:18 cslyqle variation within 75% exact homology. Variations include natural and modified amino acids.

The rare molecule fragments of insulin can be used to measure the peptidases acting on insulin based on formation of fragments. This includes the list of natural known peptidase and others added to the biological system. Additionally, rare molecule fragments of insulin can be used to measure inhibitor for peptidases acting on insulin peptidases based on the lack of formation of fragments. These inhibitors include the c-Terminal fragment of the Adiponectin Receptor, Bikunin, Uristatin and other known natural and synthetic inhibitors of archaelysin, duodenase, calpain-1, ammodytase subfamily M12B peptidases, ALE1 peptidase, CDF peptidase, cathepsin E, meprin alpha subunit, jerdohagin (Trimeresurus jerdonii), carboxypeptidase E, dibasic processing endopeptidase, yapsin-1, yapsin A, PCSK1 peptidase, aminopeptidase B, PCSK1 peptidase, PCSK2 peptidase, insulysin, and matrix metallopeptidase-9 listed in the inhibitor databases.

Rare molecule fragments of bioactive proteins and peptides can be used to measure presence or absence thereof as an indication of therapeutic effectiveness, stability, usage, metabolism, action on biological pathways (such as actions with proteases, peptidase, enzymes, receptors or other biomolecules), action of inhibition of pathways and other interactions with biological systems. Examples include but are not limited to those listed in databases of approved therapeutic peptides and proteins, such as http://crdd.osdd.net/, as well as other databases of peptides and proteins for dietary supplements, probiotics, food safety, veterinary products, and cosmetics usage. The list of the 467 approved peptide and protein therapies include examples of bioactive proteins and peptides for use in cancer, metabolic disorders, hematological disorders, immunological disorders, genetic disorders, hormonal disorders, bone disorders, cardiac disorders, infectious disease, respiratory disorders, neurological disorders, adjunct therapy, eye disorders, and malabsorption disorder. Bioactive proteins and peptides include those used as anti-thrombins, fibrinolytic, enzymes, antineoplastic agents, hormones, fertility agents, immunosuppressive agents, bone related agents, antidiabetic agents, and antibodies.

Some specific examples of therapeutic proteins and peptides include glucagon, ghrelin, leptin, growth hormone, prolactin, human placental, lactogen, luteinizing hormone, follicle stimulating hormone, chorionic gonadotropin, thyroid stimulating hormone, adrenocorticotropic hormone, vasopressin, oxytocin, angiotensin, parathyroid hormone, gastrin, buserelin, antihemophilic factor, pancrelipase, insulin, insulin aspart, porcine insulin, insulin lispro, insulin isophane, insulin glulisine, insulin detemir, insulin glargine, immunglobulins, interferon, leuprolide, denileukin, asparaginase, thyrotropin, alpha-1-proteinase inhibitor, exenatide, albumin, coagulation factors, alglucosidase alfa, salmon calcitonin, vasopressin, epidermal growth factor (EGF), cholecystokinin (CCK-8), vaccines, human growth hormone and others. Some new examples of therapeutic proteins and peptides include GLP-1-GCG, GLP-1-GIP, GLP-1, GLP-1-GLP-2, and GLP-1-CCKB

Rare molecules of interest that are highly expressed by adipose tissue include but are not limited to ADIPOQ Adiponectin, CIO and collagen domain containing, TUSC5 Tumor suppressor candidate 5, LEP Leptin, CIDEA Cell death-inducing DFFA-like effector a, CIDEC Cell death-inducing DFFA-like effector C, FABP4 Fatty acid binding protein 4, adipocyte, LIPE, GYG2, PLIN1 Perilipin 1, PLIN4 Perilipin 4, CSN1S1, PNPLA2, RP11-407P15.2 Protein LOC100509620, L GALS12 Lectin, galactoside-binding, soluble 12, GPAM Glycerol-3-phosphate acyltransferase, mitochondrial, PR325317.1 predicted protein, ACACB Acetyl-CoA carboxylase beta, ACVR1C Activin A receptor, type IC, AQP7 Aquaporin 7, CFD Complement factor D (adipsin)m CSN1S1Casein alpha s1, FASN Fatty acid synthase GYG2 Glycogenin 2 KIF25Kinesin family member 25 LIPELipase, hormone-sensitive PNPLA2 Patatin-like phospholipase domain containing 2 SLC29A4 Solute label family 29 (equilibrative nucleoside transporter), member 4 SLC7A10 Solute label family 7 (neutral amino acid transporter light chain, asc system), member 10, SPX Spexin hormone and TIMP4 TIMP metallopeptidase inhibitor 4.

Rare molecules of interest that are highly expressed by adrenal gland and thyroid include but are not limited to CYP11B2 Cytochrome P450, family 11, subfamily B, polypeptide 2, CYP11B1 Cytochrome P450, family 11, subfamily B, polypeptide 1, CYP17A1 Cytochrome P450, family 17, subfamily A, polypeptide 1, MC2R Melanocortin 2 receptor (adrenocorticotropic hormone), CYP21A2 Cytochrome P450, family 21, subfamily A, polypeptide 2, HSD3B2 Hydroxy-delta-5-steroid dehydrogenase, 3 beta- and steroid delta-isomerase 2, TH Tyrosine hydroxylase, AS3MT Arsenite methyltransferase, CYP11A1 Cytochrome P450, family 11, subfamily A, polypeptide 1, DBH Dopamine beta-hydroxylase (dopamine beta-monooxygenase), HSD3B2 Hydroxy-delta-5-steroid dehydrogenase, 3 beta- and steroid delta-isomerase 2, TH Tyrosine hydroxylase, AS3MT Arsenite methyltransferase, CYP11A1 Cytochrome P450, family 11, subfamily A, polypeptide 1, DBH Dopamine beta-hydroxylase (dopamine beta-monooxygenase), AKR1B1 Aldo-keto reductase family 1, member B1 (aldose reductase), NOV Nephroblastoma overexpressed, FDX1 Ferredoxin 1, DGKK Diacylglycerol kinase, kappa, MGARP Mitochondria-localized glutamic acid-rich protein, VWA5B2 Von Willebrand factor A domain containing 5B2, C18orf42 Chromosome 18 open reading frame 42, KIAA1024, MAP3K15 Mitogen-activated protein kinase kinase kinase 15, STAR Steroidogenic acute regulatory protein Potassium channel, subfamily K, member 2, NOV nephroblastoma overexpressed, PNMT phenylethanolamine N-methyltransferase, CHGB chromogranin B (secretogranin 1), and PHOX2A paired-like homeobox 2a.

Rare molecules of interest that are highly expressed by bone marrow include but are not limited to DEFA4 defensin alpha 4 corticostatin, PRTN3 proteinase 3, AZU1 azurocidin 1, DEFA1 defensin alpha 1, ELANE elastase, neutrophil expressed, DEFA1B defensin alpha 1B, DEFA3 defensin alpha 3 neutrophil-specific, MS4A3 membrane-spanning 4-domains, subfamily A, member 3 (hematopoietic cell-specific), RNASE3 ribonuclease RNase A family 3, MPO myeloperoxidase, HBD hemoglobin, delta, and PRSS57 serine protease 57.

Rare molecules of interest that are highly expressed by the brain include but are not limited to GFAP glial fibrillary acidic protein, OPALIN oligodendrocytic myelin paranodal and inner loop protein, OLIG2 oligodendrocyte lineage transcription factor 2, GRIN1glutamate receptor ionotropic, N-methyl D-aspartate 1, OMG oligodendrocyte myelin glycoprotein, SLC17A7 solute label family 17 (vesicular glutamate transporter), member 7, C1orf61 chromosome 1 open reading frame 61, CREG2 cellular repressor of E1A-stimulated genes 2, NEUROD6 neuronal differentiation 6, ZDHHC22 zinc finger DHHC-type containing 22, VSTM2B V-set and transmembrane domain containing 2B, and PMP2 peripheral myelin protein 2.

Rare molecules of interest that are highly expressed by the endometrium, ovary, or placenta include but are not limited to MMP26 matrix metallopeptidase 26, MMP10 matrix metallopeptidase 10 (stromelysin 2), RP4-559A3.7 uncharacterized protein and TRH thyrotropin-releasing hormone.

Rare molecules of of interest that are highly expressed by gastrointestinal tract, salivary gland, esophagus, stomach, duodenum, small intestine, or colon include but are not limited to GKN1 Gastrokine 1, GIF Gastric intrinsic factor (vitamin B synthesis), PGA5 Pepsinogen 5 group I (pepsinogen A), PGA3 Pepsinogen 3, group I (pepsinogen A, PGA4 Pepsinogen 4 group I (pepsinogen A), LCT Lactase, DEFA5 Defensin, alpha 5 Paneth cell-specific, CCL25 Chemokine (C—C motif) ligand 25, DEFA6 Defensin alpha 6 Paneth cell-specific, GAST Gastrin, MS4A10 Membrane-spanning 4-domains subfamily A member 10, ATP4A and ATPase, H+/K+ exchanging alpha polypeptide.

Rare molecules of of interest that are highly expressed by heart or skeletal muscle include but are not limited to NPPB natriuretic peptide B, TNNI3 troponin I type 3 (cardiac), NPPA natriuretic peptide A, MYL7 myosin light chain 7 regulatory, MYBPC3 myosin binding protein C (cardiac), TNNT2 troponin T type 2 (cardiac) LRRC10 leucine rich repeat containing 10, ANKRD1 ankyrin repeat domain 1 (cardiac muscle), RD3L retinal degeneration 3-like, BMP10 bone morphogenetic protein 10, CHRNE cholinergic receptor nicotinic epsilon (muscle), and SBK2 SH3 domain binding kinase family member 2.

Rare molecules of of interest that are highly expressed by kidney include but are not limited to UMOD uromodulin, TMEM174 transmembrane protein 174, SLC22A8 solute label family 22 (organic anion transporter) member 8, SLC12A1 solute label family 12 (sodium/potassium/chloride transporter) member 1, SLC34A1 solute label family 34 (type II sodium/phosphate transporter) member 1, SLC22A12 solute label family 22 (organic anion/urate transporter) member 12, SLC22A2 solute label family 22 (organic cation transporter) member 2, MCCD1 mitochondrial coiled-coil domain 1, AQP2 aquaporin 2 (collecting duct), SLC7A13 solute label family 7 (anionic amino acid transporter) member 13, KCNJ1 potassium inwardly-rectifying channel, subfamily J member 1 and SLC22A6 solute label family 22 (organic anion transporter) member 6.

Rare molecules of interest that are highly expressed by lung include but are not limited to SFTPC surfactant protein C, SFTPA1 surfactant protein A1, SFTPB surfactant protein B, SFTPA2 surfactant protein A2, AGER advanced glycosylation end product-specific receptor, SCGB3A2 secretoglobin family 3A member 2, SFTPD surfactant protein D, ROS1 proto-oncogene 1 receptor tyrosine kinase, MS4A15 membrane-spanning 4-domains subfamily A member 15, RTKN2 rhotekin 2, NAPSA napsin A aspartic peptidase, and LRRN4 leucine rich repeat neuronal 4.

Rare molecules of of interest that are highly expressed by liver or gallbladder include but are not limited to APOA2 apolipoprotein A-II, A1BG alpha-1-B glycoprotein, AHSG alpha-2-HS-glycoprotein, F2coagulation factor II (thrombin), CFHR2 complement factor H-related 2, HPX hemopexin, F9 coagulation factor IX, CFHR2 complement factor H-related 2, SPP2 secreted phosphoprotein 2 (24 kDa), C9 complement component 9, MBL2 mannose-binding lectin (protein C) 2 soluble and CYP2A6 cytochrome P450 family 2 subfamily A polypeptide 6. Rare molecules of of interest that are highly expressed by testis or prostate include but are not limited to PRM2 protamine 2 PRM1 protamine 1 TNP1 transition protein 1 (during histone to protamine replacement) TUBA3C tubulin, alpha 3c LELP1late cornified envelope-like proline-rich 1 BOD1L2 biorientation of chromosomes in cell division 1-like 2 ANKRD7 ankyrin repeat domain 7 PGK2 phosphoglycerate kinase 2 AKAP4 A kinase (PRKA) anchor protein 4 TPD52L3 tumor protein D52-like 3 UBQLN3 ubiquilin 3 and ACTL7A actin-like 7A.

### Examples of Rare Cells and Rare Cell Markers

Rare cells are those cells that are present in a sample in relatively small quantities when compared to the amount of non-rare cells in a sample. In some examples, the rare cells are present in an amount of about 10⁻⁸% to about 10⁻²% of the total cell population in a sample suspected of containing the rare cells. The phrase “cellular rare molecules” refers to rare molecules that are bound in a cell and may or may not freely circulate in a sample. Such cellular rare molecules include biomolecules useful in medical diagnosis of diseases as above and also include all rare molecules and uses previously described for cell free rare molecules and those for biomolecules used for measurement of rare cells. The rare cells (cell markers) may be, but are not limited to, malignant cells such as malignant neoplasms or cancer cells; circulating cells, endothelial cells (CD146); epithelial cells (CD326/EpCAM); mesochymal cells (VIM), bacterial cells, virus, skin cells, sex cells, fetal cells; immune cells (leukocytes such as basophil, granulocytes (CD66b) and eosinophil, lymphocytes such as B cells (CD19,CD20), T cells (CD3,CD4 CD8), plasma cells, and NK cells (CD56), macrophages/monocytes (CD14, CD33), dendritic cells (CD11c, CD123), Treg cells and others), stem cells/precursor (CD34), other blood cells such as progenitor, blast, erythrocytes, thrombocytes, platelets (CD41, CD61, CD62) and immature cells; other cells from tissues such as liver, brain, pancreas, muscle, fat, lung, prostate, kidney, urinary tract, adipose, bone marrow, endometrium, gastrointestinal tract, heart, testis or others.

The phrase “population of cells” refers to a group of cells having an antigen or nucleic acid marker on their surface or inside the cell where the marker is common to all of the cells of the group and where the marker is specific for the group of cells. Non-rare cells are those cells that are present in relatively large amounts when compared to the amount of rare cells in a sample. In some examples, the non-rare cells are at least about 10 times, or at least about 10² times, or at least about 10³ times, or at least about 10⁴ times, or at least about 10⁵ times, or at least about 10⁶ times, or at least about 10⁷ times, or at least about 10⁸ times greater than the amount of the rare cells in the total cell population in a sample suspected of containing non-rare cells and rare cells. The non-rare cells may be, but are not limited to, white blood cells, platelets, and red blood cells.

The term “rare cells markers” describe markers that include, but are not limited to, cancer cell type biomarkers, cancer biomarkers, chemo resistance biomarkers, metastatic potential biomarkers, and cell typing markers, cluster of differentiation (cluster of designation or classification determinant) (often abbreviated as CD, a protocol used for the identification and investigation of cell surface molecules providing targets for immunophenotyping of cells), for example. Cancer cell type biomarkers include, by way of illustration and not limitation, cytokeratins (CK) (CK1, CK2, CK3, CK4, CK5, CK6, CK7, CK8 and CK9, CK10, CK12, CK 13, CK14, CK16, CK17, CK18, CK19 and CK20), epithelial cell adhesion molecule (EpCAM), N-cadherin, E-cadherin and vimentin. Oncoproteins and oncogenes with likely therapeutic relevance due to mutations include, but are not limited to, WAF, BAX-1, PDGF, JAGGED 1, NOTCH, VEGF, VEGHR, CA1X, MIB1, MDM, PR, ER, SELS, SEMI, PI3K, AKT2, TWIST1, EML-4, DRAFF, C-MET, ABL1, EGFR, GNAS, MLH1, RET, MEK1, AKT1, ERBB2, HER2, HNF1A, MPL, SMAD4, ALK, ERBB4, HRAS, NOTCH1, SMARCB1, APC, FBXW7, IDHL NPM1, SMO, ATM, FGFR1, JAK2, NRAS, SRC, BRAF, FGFR2, JAK3, RA, STK11, CDH1, FGFR3, KDR, PIK3CA, TP53, CDKN2A, FLT3, KIT, PTEN, VHL, CSF1R, GNA11, KRAS, PTPN11, DDR2, CTNNB1, GNAQ, MET, RB1, AKT1, BRAF, DDR2, MEK1, NRAS, FGFR1, and ROS1.

In certain embodiments, the rare cells may be endothelial cells which are detected using markers, by way of illustration and not limitation, CD136, CD105/Endoglin, CD144/VE-cadherin, CD145, CD34, Cd41 CD136, CD34, CD90, CD31/PECAM-1, ESAM,VEGFR2/Fik-1, Tie-2, CD202b/TEK, CD56/NCAM, CD73/VAP-2, claudin 5, ZO-1, and vimentin. Metastatic potential biomarkers include, but are limited to, urokinase plasminogen activator (uPA), tissue plasminogen activator (tPA), C terminal fragment of adiponectin receptor (Adiponectin Receptor C Terminal Fragment or Adiponectin CTF), kinases (AKT-PIK3, MAPK), vascular adhesion molecules (e.g., ICAM, VCAM, E-selectin), cytokine signaling (TNF-α, IL-1, IL-6), reactive oxidative species (ROS), protease-activated receptors (PARs), metalloproteinases (TIMP), transforming growth factor (TGF), vascular endothelial growth factor (VEGF), endothelial hyaluronan receptor 1 (LYVE-1), hypoxia-inducible factor (HIF), growth hormone (GH), insulin-like growth factors (IGF), epidermal growth factor (EGF), placental growth factor (PDF), hepatocyte growth factor (HGF), nerve growth factor (NGF), platelet-derived growth factor (PDGF), growth differentiation factors (GDF), VEGF receptor (soluble Flt-1), microRNA (MiR-141), Cadherins (VE, N, E), S100 Ig-CTF nuclear receptors (e.g., PPARa), plasminogen activator inhibitor (PAI-1), CD95, serine proteases (e.g., plasmin and ADAM, for example); serine protease inhibitors (e.g., Bikunin); matrix metalloproteinases (e.g., MMP9); matrix metalloproteinase inhibitors (e.g., TIMP-1); and oxidative damage of DNA.

Chemoresistance biomarkers include, by way of illustration and not limitation, PL2L piwi like, 5T4, ADLH, β-integrin, α-6-integrin, c-kit, c-met, LIF-R, chemokines (e.g., CXCR7,CCR7, CXCR4), ESA, CD 20, CD44, CD133, CKS, TRAF2 and ABC transporters, cancer cells that lack CD45 or CD31 but contain CD34 are indicative of a cancer stem cell; and cancer cells that contain CD44 but lack CD24.

The rare molecules from cells may be from any organism, including but not limited to, pathogens such as bacteria, virus, fungus, and protozoa; malignant cells such as malignant neoplasms or cancer cells; circulating endothelial cells; circulating tumor cells; circulating cancer stem cells; circulating cancer mesochymal cells; circulating epithelial cells; fetal cells; immune cells (B cells, T cells, macrophages, NK cells, monocytes); and stem cells; for example. In some examples of methods in accordance with the principles described herein, the sample to be tested is a blood sample from a mammal such as, but not limited to, a human subject.

Rare cells of interest may be immune cells and include but are not limited to markers for white blood cells (WBC), Tregs (regulatory T cells), B cell, T cells, macrophages, monocytes, antigen presenting cells (APC), dendritic cells, eosinophils, and granulocytes. For example, markers such as, but not limited to, CD3, CD4, CD8, CD11c, CD14, CD15, CD16, CD19, CD20, CD31, CD33, CD45, CD52, CD56, CD 61, CD66b, CD123, CTLA-4, immunoglobulin, protein receptors and cytokine receptors and other CD markers that are present on white blood cells can be used to indicate that a cell is not a rare cell of interest.

In a particular non-limiting examples white blood cell markers include CD45 antigen (also known as protein tyrosine phosphatase receptor type C or PTPRC), which is originally called leukocyte common antigen and is useful in detecting all white blood cells. Additionally, CD45 can be used to differentiate different types of white blood cells that might be considered rare cells. For example, granulocytes are indicated by CD45+, CD15+, or CD16+, or CD66b+; monocytes are indicated by CD45+, CD14+; T lymphocytes are indicated by CD45+, CD3+; T helper cells are indicated by CD45+,CD3+, CD4+; cytotoxic T cells are indicated by CD45+,CD3+, CDS+; B-lymphocytes are indicated by CD45+, CD19+, or CD45+, CD20+; thrombocytes are indicated by CD45+, CD61+; and natural killer cells are indicated by CD16+, CD56+, and CD3−. Furthermore, two commonly used CD molecules, namely, CD4 and CD8, are, in general, used as markers for helper and cytotoxic T cells, respectively. These molecules are defined in combination with CD3+, as some other leukocytes also express these CD molecules (some macrophages express low levels of CD4). Dendritic cells express high levels of CD11c, and CD123. These examples are not inclusive of all marker and are for example purposes only.

In some cases, the rare molecule fragments of lymphocytes include proteins and peptides produced as part of lymphocytes such as immunoglobulin chains, major histocompatibility complex (MHC) molecules, T cell receptors, antigenic peptides, cytokines, chemokines and their receptors (e.g, Interluekins, C—X—C chemokine receptors, etc), programmed death-ligand and receptors (Fas, PDL1, and others), and other proteins and peptides that are either parts of the lymphocytes or bind to the lymphocytes.

In other cases the rare cell maybe a stem cell. Rare molecule markers of stem cells include, but are not limited to, PL2L piwi like, 5T4, ADLH, β-integrin, α6 integrin, c-kit, c-met, LIF-R, CXCR4, ESA, CD 20, CD44, CD133, CKS, TRAF2 and ABC transporters, cancer cells that lack CD45 or CD31 but contain CD34 indicative of a cancer stem cell; and cancer cells that contain CD44 but lack CD24. Stem cell markers include common pluripotency markers like FoxD3, E-Ras, Sall4, Stat3, SUZ12, TCF3, TRA-1-60, CDX2, DDX4, Miwi, Mill GCNF, Oct4, Klf4, Sox2,c-Myc, TIF1, Piwil1-4, nestin, integrin, notch, AML, GATA, Esrrb, Nr5a2, C/EBPa, Lin28, Nanog, insulin, neuroD, adiponectin, apdiponectin receptor, FABP4, PPAR, and KLF4 and the like.

In other cases the rare cell maybe a pathogen, bacteria, or virus or group thereof which includes, but is not limited to, gram-positive bacteria (e.g., Enterococcus sp. Group B streptococcus, Coagulase-negative staphylococcus sp., Streptococcus viridans, Staphylococcus aureus and saprophyicus, Lactobacillus and resistant strains thereof, for example); yeasts including, but not limited to, Candida albicans, for example; gram-negative bacteria such as, but not limited to, Escherichia coli, Klebsiella pneumoniae, Citrobacter koseri, Citrobacter freundii, Klebsiella oxytoca, Morganella morganii, Pseudomonas aeruginosa, Proteus mirabilis, Serratia marcescens, Diphtheroids (gnb), Rosebura, Eubacterium hallii, Faecalibacterium prauznitzli, Lactobacillus gasseria, Streptococcus mutans, Bacteroides thetaiotaomicron, Prevotella Intermedia, Porphyromonas gingivalis Eubacterium rectale Lactobacillus amylovorus, Bacillus subtilis, Bifidobacterium longum, Eubacterium rectale, E. eligens, E. dolichum, B. thetaiotaomicron, E. rectale, Actinobacteria, Proteobacteria, B. thetaiotaomicron, Bacteroides Eubacterium dolichum, Vulgatus, B. fragilis, bacterial phyla such as Firmicuties (Clostridia, Bacilli, Mollicutes), Fusobacteria, Actinobacteria, Cyanobacteria, Bacteroidetes, Archaea, Proteobacteria, and resistant strains thereof, for example; viruses such as, but not limited to, HIV, HPV, Flu, and MERSA, for example; and sexually transmitted diseases. In the case of detecting rare cell pathogens, a particle reagent is added that comprises a binding partner, which binds to the rare cell pathogen population. Additionally, for each population of cellular rare molecules on the pathogen, a reagent is added that comprises a binding partner for the cellular rare molecule, which binds to the cellular rare molecules in the population.

As mentioned above, some examples in accordance with the principles described herein are directed to methods of detecting a cell, which include natural and synthetic cells. The cells are usually from a biological sample that is suspected of containing target rare molecules, non-rare cells and rare cells. The samples may be biological samples or non-biological samples. Biological samples may be from a mammalian subject or a non-mammalian subject. Mammalian subjects may be, e.g., humans or other animal species.

### Examples of Apparatus and Reagents for Conducting Methods

The apparatus and reagents for conducting a method in accordance with the principles described herein may be present in a kit useful for conveniently performing the method. In one embodiment, a kit comprises combination of affinity agents, each one for a different rare molecule to be isolated. The kit may also comprise one or more cell affinity agents for cells containing the rare molecules, the porous matrix, optional capture particles, solutions for spraying, filtering and releasing the mass labels, a droplet generator, capillary nozzles for droplet formation, capillary channels for dilution, concentration or routing of solutions, droplets and molecules, solutions for forming droplets, solutions for breaking droplets. The composition may contain label particles or capture particle entities, for example, as described above. Porous matrix, liquid holding wells, porous matrix and droplet generators can be in housing where the housing can have vents, capillaries, chambers, liquid inlets and outlets. A solvent can be applied to droplet generators, wells and porous matrix. Porous matrix can be removeable.

Depending on the method for analysis of selected rare molecules, reagents discussed in more detail herein below, may or may not be used to treat the samples during, prior to or after the extraction of molecules from the rare cells and cell free samples.

The concentrations of the various reagents in the kits can be varied widely to allow substantial optimization of the reactions that need to occur during the present methods and to further allow optimizing substantially for the sensitivity of the methods. Under appropriate circumstances one or more of the reagents in the kit can be provided as a dry powder, usually lyophilized, including excipients, which on dissolution will provide for a reagent solution having the appropriate concentrations for performing a method in accordance with the principles described herein. The kit can further include a written description of a method utilizing reagents in accordance with the principles described herein.

The phrase “at least” as used herein means that the number of specified items may be equal to or greater than the number recited. The phrase “about” as used herein means that the number recited may differ by plus or minus 10%; for example, “about 5” means a range of 4.5 to 5.5.

The spray solvent can be any spray solvent employed in electrospray mass spectroscopy. In some examples, solvents for electrospray ionization include, but are not limited to, polar organic compounds such as, e.g., alcohols (e.g., methanol, ethanol and propanol), acetonitrile, dichloromethane, dichloroethane, tetrahydrofuran, dimethylformamide, dimethyl sulphoxide, and nitromethane; non-polar organic compounds such as, e.g., hexane, toluene, cyclohexane; and water, for example, or combinations of two or more thereof. Optionally, the solvents may contain one or more of an acid or a base as a modifier (such as, volatile salts and buffer, e.g., ammonium acetate, ammonium biocarbonate, volatile acids such as formic acid, acetic acids or trifluoroacetic acid, heptafluorobutyric acid, sodium dodecyl sulphate, ethylenediamine tetraacetic acid, and non-volatile salts or buffers such as, e.g., chlorides and phosphates of sodium and potassium, for example.

In many examples, the sample is in contact with an aqueous phase prior to forming an emulsion. The aqueous phase may be solely water or that which may also contain organic solvents such as, for example, polar aprotic solvents, polar protic solvents such as, e.g., dimethylsulfoxide (DMSO), dimethylformamide (DMF), acetonitrile, an organic acid, or an alcohol, and non-polar solvents miscible with water such as, e.g., dioxene, in an amount of about 0.1% to about 90%, by volume. In some examples, the pH for the aqueous medium is usually a moderate pH. In some examples, the pH of the aqueous medium is about 5 to about 8, or about 6 to about 8, or about 7 to about 8, or about 5 to about 7, or about 6 to about 7, or physiological pH. Various buffers may be used to achieve the desired pH and maintain the pH during any incubation period. Illustrative buffers include, but are not limited to, borate, phosphate (e.g., phosphate buffered saline), carbonate, TRIS, barbital, PIPES, HEPES, IVIES, ACES, MOPS, and BICINE.

Cell and/or droplet lysis reagents are those that involve disruption of the integrity of the cellular membrane with a lytic agent, thereby releasing intracellular contents of the cells. Numerous lytic agents are known in the art. Lytic agents that may be employed may be physical and/or chemical agents. Physical lytic agents include, blending, grinding, and sonication, and combinations of two or more thereof, for example. Chemical lytic agents include, but are not limited to, non-ionic detergents, anionic detergents, amphoteric detergents, low ionic strength aqueous solutions (hypotonic solutions), bacterial agents, and antibodies that cause complement dependent lysis, and combinations of two or more thereof, for example, and combinations or two or more of the above. Non-ionic detergents that may be employed as the lytic agent include both synthetic detergents and natural detergents.

The nature and amount or concentration of lytic agent employed depends on the nature of the cells, the nature of the cellular contents, the nature of the analysis to be carried out, and the nature of the lytic agent, for example. The amount of the lytic agent is at least sufficient to cause lysis of cells to release contents of the cells. In some examples the amount of the lytic agent is about 0.0001% to about 5% (percentages are by weight), for example.

Removal of lipids, platelets, and non rare cells may be carried out using, by way of illustration and not limitation, detergents, surfactants, solvents, and binding agents, and combinations of two or more of the above, for example, and combinations of two or more thereof. The use of a surfactant or a detergent as a lytic agent as discussed above accomplishes both cell lysis and removal of lipids. The amount of the agent for removing lipids is at least sufficient to remove at least about 50%, or at least about 60%, or at least about 70%, or at least about 80%, or at least about 90%, or at least about 95% of lipids from the cellular membrane. In some examples the amount of the lytic agent is about 0.0001% to about 5% (percentages by weight), for example.

In some examples, it may be desirable to remove or denature proteins from the cells, which may be accomplished by using a proteolytic agent such as, but not limited to, proteases, heat, acids, phenols, and guanidinium salts, and combinations of two or more thereof, for example. The amount of the proteolytic agent is at least sufficient to degrade at least about 50%, or at least about 60%, or at least about 70%, or at least about 80%, or at least about 90%, or at least about 95% of proteins in the cells. In some examples the amount of the proteolytic agent is about 0.0001% to about 5% (percentages by weight), for example.

In some examples, samples are collected from the body of a subject into a suitable container such as, but not limited to, a cup, a bag, a bottle, capillary, or a needle, for example. Blood samples may be collected into VACUTAINER® containers, for example. The container may contain a collection medium into which the sample is delivered. The collection medium is usually a dry medium and may comprise an amount of platelet deactivation agent effective to achieve deactivation of platelets in the blood sample when mixed with the blood sample.

Platelet deactivation agents can be added to the sample such as, but not limited to, chelating agents such as, for example, chelating agents that comprise a triacetic acid moiety or a salt thereof, a tetraacetic acid moiety or a salt thereof, a pentaacetic acid moiety or a salt thereof, or a hexaacetic acid moiety or a salt thereof. In some examples, the chelating agent is ethylene diamine tetraacetic acid (EDTA) and its salts or ethylene glycol tetraacetate (EGTA) and its salts. The effective amount of platelet deactivation agent is dependent on one or more of the following, the nature of the platelet deactivation agent, the nature of the blood sample, level of platelet activation and ionic strength, for example. In some examples, with EDTA as the anti-platelet agent, the amount of dry EDTA in the container is that which will produce a concentration of about 1.0 to about 2.0 mg/mL of blood, or about 1.5 mg/mL of the blood. The amount of the platelet deactivation agent is that which is sufficient to achieve at least about 90%, or at least about 95%, or at least about 99% of platelet deactivation.

Temperatures employed in the methods may range from about 5° C. to about 70° C. or from about 15° C. to about 70° C. or from about 20° C. to about 45° C., or from about 55° C. to about 95° C. for example. The time period for an incubation period is about 0.2 seconds to about 6 hours, or about 2 seconds to about 1 hour, or about 1 to about 5 minutes, for example. These temperatures can be used to reverse fixations, deactivate nucleases or proteases, or for other purposes.

In many examples, the above combination is provided in an aqueous medium, which may be solely water or which may also contain organic solvents such as, for example, polar aprotic solvents, polar protic solvents such as, e.g., dimethylsulfoxide (DMSO), dimethylformamide (DMF), acetonitrile, an organic acid, or an alcohol, and non-polar solvents miscible with water such as, e.g., dioxene, in an amount of about 0.1% to about 50%, by volume. In some examples, the pH for the aqueous medium is usually a moderate pH. In some examples the pH of the aqueous medium is about 5 to about 8, or physiological pH, for example. Various buffers may be used to achieve the desired pH and maintain the pH during any incubation period. Illustrative buffers include, but are not limited to, borate, phosphate (e.g., phosphate buffered saline), carbonate, TRIS, barbital, PIPES, HEPES, MES, ACES, MOPS, and BICINE, for example.

An amount of aqueous medium employed is dependent on a number of factors such as, but not limited to, the nature and amount of the sample, the nature and amount of the reagents, the stability of rare cells, and the stability of rare molecules, for example. In some examples in accordance with the principles described herein, the amount of aqueous medium per 10 mL of sample is about 1 mL to about 100 mL for example.

Where one or more of the rare nucleic acids are part of a cell, the aqueous medium may also comprise a lysing agent for lysing of cells. A lysing agent is a compound or mixture of compounds that disrupt the integrity of the matrixes of cells thereby releasing intracellular contents of the cells. Examples of lysing agents include, but are not limited to, non-ionic detergents, anionic detergents, amphoteric detergents, low ionic strength aqueous solutions (hypotonic solutions), bacterial agents, aliphatic aldehydes, and antibodies that cause complement dependent lysis, for example. Various ancillary materials may be present in the dilution medium. All of the materials in the aqueous medium are present in a concentration or amount sufficient to achieve the desired effect or function.

In some examples, it may be desirable to fix the nucleic acids, proteins or cells of the sample. Fixation immobilizes the nucleic acids and preserves the nucleic acids structure and maintains the cells in a condition that closely resembles the cells in an in vivo-like condition and one in which the antigens of interest are able to be recognized by a specific affinity agent. The amount of fixative employed is that which preserves the nucleic acids or cells but does not lead to erroneous results in a subsequent assay. The amount of fixative depends on one or more of the following, the nature of the fixative and the nature of the cells, for example. In some examples, the amount of fixative is about 0.05% to about 0.15% or about 0.05% to about 0.10%, or about 0.10% to about 0.15% by weight. Agents for carrying out fixation of the cells include, but are not limited to, cross-linking agents such as, for example, an aldehyde reagent (such as, e.g., formaldehyde, glutaraldehyde, and paraformaldehyde,); an alcohol (such as, e.g., C₁-C₅ alcohols such as methanol, ethanol and isopropanol); a ketone (such as a C₃-C₅ ketone such as acetone); for example. The designations C₁-C₅ or C₃-C₅ refer to the number of carbon atoms in the alcohol or ketone. One or more washing steps may be carried out on the fixed cells using a buffered aqueous medium.

In examples in which fixation is employed, extraction of nucleic acids can include a procedure for de-fixation prior to amplification. De-fixation may be accomplished employing, by way of illustration and not limitation, heat or chemicals capable of reversing cross-linking bonds, or a combination of both.

In some examples utilizing the techniques, it may be necessary to subject the rare cells to permeabilization. Permeabilization provides access through the cell membrane to antigens or nucleic acids of interest. The amount of permeabilization agent employed is that which disrupts the cell membrane and permits access to the antigens or nucleic acids. The amount of permeabilization agent depends on one or more of the nature of the permeabilization agent and the nature and amount of the rare cells, for example. In some examples, the amount of permeabilization agent by weight is about 0.01% to about 0.5%, for example. Agents for carrying out permeabilization of the rare cells include, but are not limited to, an alcohol (such as, e.g., C₁-C₅ alcohols such as methanol and ethanol); a ketone (such as a C₃-C₅ ketone such as acetone); a detergent (such as, e.g., saponin, Triton® X-100, and Tween®-20); for example. One or more washing steps may be carried out on the permeabilized cells using a buffered aqueous medium.

The following examples further describe the specific embodiments of the invention by way of illustration and not limitation and are intended to describe and not to limit the scope of the invention. Parts and percentages disclosed herein are by volume unless otherwise indicated.

### EXAMPLES

All chemicals may be purchased from the Sigma-Aldrich Company (St. Louis, Mo.) unless otherwise noted.

### Abbreviations

K₃EDTA=potassium salt of ethylenediaminetetraacetate
min=minute(s)
=micron(s)
mL=milliliter(s)
mg=milligrams(s)
=microgram(s)
PBS=phosphate buffered saline (3.2 mM Na₂HPO₄, 0.5 mM KH₂PO₄, 1.3 mM KCl, 135 mM NaCl, pH 7.4)
mBar=millibar
w/w=weight to weight
RT=room temperature
hr=hour(s)
QS=quantity sufficient
Ab=antibody
mAb=monoclonal antibody
vol=volume
MW=molecular weight
wt.=weight
Transfix® tube=10 mL Vacutest Kima blood collection tube containing K₃EDTA and 0.45 mL Transfix®
SKBR cells=SKBR3 human breast cancer cells (ATCC)
Affinity agent for Her2nue on capture particle=Her2nue obtained from lyzed SKBR3 human breast cancer cells (TA1 clone) (ATCC)
Affinity agent for Her2nue on label particle=Monoclonal anti Her2nue antibody (NB3 clone) (ATCC)
WBC=white blood cells
Lysis buffer=5M buffered guanidine thiocyanate, detergent
Blocking agent=Casien, the blocking solution (Candor Biosience GmbH, Allgau Germany)
Capture particle with a specific nucleic acid affinity agent=Magnetic beads with streptavidin bond to a specific nucleic acid affinity agent through a biotin
Antigen capture particles=BioMag® hydroxyl silica micro particles (46.2 mg/mL, 1.5 μm) with streptavidin (Bangs Lab Inc.) with anti Her2nue antibody (NB3 clone from ATCC) made by direct conjugation to the particles.
Label particle=Propylamine-functionalized silica nano-particles 200 μm, mesoporous pore sized 4 nm with the SPDP-modification to linke SH-peptide/SH-neutravidin byt a disulfide bond to the silica amine label particle.
Nucleic acid capture particles=poly T or CK19 hybridization oligo bound to biotin and mixed with BioMag® hydroxyl silica micro particles (2.0 mg/mL, 1.5 μm) with streptavidin (Bangs Lab Inc.)
Porous Matrix=WHATMAN® NUCLEOPORE™ Track Etch matrix, 25 mm diameter and 8.0 and 1.0 μM pore sizes
Wash buffer=Phosphate buffered saline (PBS) with 0.2% TWEEN® 20 surfactant
Elution buffer=25 mM Tris-HCl, pH 8 buffer for non-selective extraction and 25 mM citrate pH 3.1 buffer for selective extraction
Cell affinity agents=cytokeratin 8/18 antibody attached to biotin which specifically binds to SBKR cells.
Proteolytic buffer=25 mM Tris-NaCl, 0.3% proteinase K (Invitrogen CA) DNase solution=DNase buffer (Qiagen mat#1064143, Qiagen, Inc.) and DNase I (Qiagen mat#1064141, Qiagen, Inc.).
MS=Mass spectroscopy analysis by nano electrospray ionization on a THERMO LTQ (linear ion trap) mass spectrometer (from Thermo Electron North America LLC).

### Example 1

### Method for Detection of Rare Genes and Proteins from Same Sample

An example of a method for detection of rare molecules in accordance with the principles described herein is depicted in FIGS. 1-3 and in this example, antigens and nucleic acids from a sample are captured on particles or cells. Undesired material washed way, such that antigens and nucleic acids remain captured on particles retained on a porous matrix. A measurement of the protein is obtained in a means not destructive to captured nucleic acids. The residual sample is sealed and protected from contamination. The retained nucleic acids are amplified into nucleic acid products not attached to particles for detection or removal.

The example used size exclusion filtration for retaining materials as previously described (Using Automated Microfluidic Filtration and Multiplex Immunoassay Magbanua M J M, Pugia M, Lee J S, Jabon M, Wang V, et al. (2015); A Novel Strategy for Detection and Enumeration of Circulating Rare Cell Populations in Metastatic Cancer Patients Using Automated Microfluidic Filtration and Multiplex Immunoassay. PLoS ONE 10(10)). The only change to the process was to use a vacuum filtration unit (Biotek Inc) for a standard ELISA plate fitted with the unit.

The sample was filtered through liquid holding wells typical of those of a 96-well ELISA plate. The liquid holding wells are 6.5 mm in diameter. The bottom of each well has a porous matrix. A porous matrix with 8.0 μm pores were used for cell and droplet capture, or with 1.0 μm pores for particle capture. The cells in this example were ˜20 μm in diameter (5 to 30 μm range), and nucleic acids and proteins were ˜1 to 20 μm in diameter (10 to 400 nm range). Label particles were ˜20 nm in diameter (10 to 100 nm range) and capture particles were ˜1.5 μm in diameter (1 to 2 μm range), and droplets were ˜10 μm in diameter (5 to 20 μm range).

Cells, droplets and particles were retained onto a porous matrix when subjected to a negative mBar, that is, a decrease greater than about −100 mBar from atmospheric pressure. The vacuum applied varied from −10 to −100 mBar during filtration. The diluted sample was placed into the filtration station and the sample was filtered through the porous matrix. In all cases the porous matrix was at the bottom of a well. After the liquid was removed by vacuum filtration, a surfactant, in this case 0.5% Triton X-100 in PBS was added to wash away the unbound materials. Label particles, genes and proteins that were not bound were removed.

Whole blood specimens were collected from donor or patient (˜8 mL each tube) into Transfix® tubes according to an IRB-approved protocol. Tubes were inverted 20 times and allowed to sit for 24 hours at room temperature (RT). Cellular nucleic acids were introduced by adding SKBR human breast cancer cells, in which formed a concentration of 1 to 1000 cells/tube. Whole blood aliquots of 0.5 mL were added to 2.5 mL of PBS buffer in polypropylene sterile centrifuge tubes.

The following demonstrates the general method of cellular and cell free antigen and nucleic acid retention and measurement. Cells, particles and droplets were first reacted with affinity agents, which, in the cell free case, are mAb that selectively bind to antigen and a poly-T nucleic acid probe that selectively bind to mRNA. In the cellular case, the cells or droplets are isolated on the porous matrix. In all cases, unbound nucleic acid are washed away using a series of liquids following the filtration. In this case the porous matrix was washed with PBS, and the sample was fixed with formaldehyde, washed with PBS, subjected to permeabilization using 0.2% TRITON® X-100 in PBS and washed again with PBS. A blocking step was employed in which blocking buffer of 10% casein in PBS was dispensed on the porous matrix prior to adding the cell affinity agents. After an incubation period of 5 min, the matrix was washed with PBS to block non-specific binding to the matrix. Multiple wash buffers were used to wash porous matrix after each affinity reaction. Cells, particles and droplets were then measured using affinity reactions and immunocytochemistry (ICC) with a fluorescent label attached to the antibody for antigen.

Antigens were isolated from the human blood using a capture method and unique antibodies for the Her2nue protein. For isolation of cell free Her2nue antigens, capture was done with particles (50 μL of magnetic beads) with antibodies for the Her2nue using sample with lysed SKBR cells. For isolation of cellular Her2nue antigens, capture was done using intact SKBR cells as the sample. Unbound proteins were washed away through the porous matrix. The Her2nue antigens were prepared for detection by treatment of capture particles, cells or droplets with label particles attached to a second antibody for the Her2nue protein. The labeled nanoparticles (15 to 200 nm) were also coated with a release-able analytical label, in this case a peptide attached by a sulfhydryl, and a non-releasable fluorescent label, in this case Dylight 488 attached to NeutrAvidin. The label particles were linked to capture particles by biotin-NeutrAvidin reaction and unbound label particles were washed away.

Nucleic acids were isolated from the human blood using a capture method for CK19 mRNA. For isolation of cell free CK19 mRNA molecules, capture was done with particles (50 of magnetic beads) with poly-T as an affinity agent for the CK19 mRNA using a sample containing lysed SKBR cells. For isolation of cellular CK 19 mRNA, capture was done using intact SKBR cells as the sample. Particles, droplets and cells with CK19 mRNA were retained on porous matrix. Unbound mRNA were washed away through the porous matrix. The mRNA for CK19 was captured after release of CK19 mRNA by lysis buffer in case of cellular assay and elution buffer in cell free assay, and converted to its cDNA by reverse transcriptase (RT). In this case the amplified product was not bound to the particle. The samples from selective cell nucleic acid isolation were able to achieve a minimal purity of CK19 mRNA in the range of 0.01% to about 20% and still achieve the minimal copy number 100 to about 10,000,000 minimal purity of rare nucleic acid for 10-50 SBKR cells in 0.5 mL of whole blood with all the expected nucleic acids cellular assays (See Table 1). In some examples, mRNA was amplified by RT and cDNA captured before protein analysis (See Table 1). In other examples, cell free DNA was captured and amplified after protein analysis (See Table 1). In all cases the porous matrix could be sealed to prevent contamination, the protein analysis was non-destructive and the minimal purity was achieved. In other examples the gDNA from the sample is amplified by polymerase after protein analysis and cDNA removed after amplification.

The contents retained on the porous matrix were also measured by fluorescent microscopy and digital imaging to locate the contents. Images were analyzed to identify the antigens and nucleic acids captured in the cell, droplets and particles with unbound label particles washed away. The antigens inside cells were identified by the antigen-binding antibody attached to a label particle. The label particle was modified with an analytical label and neutravidin such that a biotinylated antibody affinity agent binds the neutravidin on the label particle. A fluorescent dye, in this case dylight 550 is attached to the neutravidin. The same neutravidin on label particle could be used to bind a biotin connected to a nucleic acid affinity agent. The presence of fluorescent dye in the cell indicated the antibody affinity agent and/or the nucleic acid affinity agent bound to retained antigens and nucleic acids. This experiment demonstrated the antigens and nucleic acids were retained in droplets or by capture particles.

Isolated antigens were first treated to break the —S—S— bond and release the analytical label from the label particle. The sample was treated with 10 μL of a TCEP solution (1 mg/mL in 50% ACN/H2O) to release the analytical label. Analysis by mass spectroscopy (MS) demonstrated>90% capture and release efficiencies of this process by comparison to know amount of analytical labels added. A peptide analytical label was used for mass spectroscopy (MS) quantification of the amount of Her2nue antigen. A series of experiments was performed to calculate analytical sensitivity of detecting cell and cell-free Her2nue antigens, and the CK19 mRNA in a whole blood sample. The observed analytical sensitivity was determined by measurements of samples with 0 to 1000 intact or lysed SKBR cells added to whole blood. Additionally, the cell and cell-free limits of detection were comparable to the typical limit of detection of 50 cells and are reported in Table 1.

The procedure to amplify and analyze nucleic acids isolated was demonstrated with mRNA for CK19 sequence as a disease-related rare nucleic acid and a reverse-transcription quantitative PCR (RT-qPCR) after the samples of nucleic acid were selectively enriched in cell-free or cellular studies. The enriched cell-free RNA was removed from the porous matrix by placing the porous matrix in a 1.5 mL tube and the porous matrix was pushed to the bottom of the tube using forceps and combined with 50 μL of lysis buffer containing a protease to release RNA from cells. The tubes were incubated at 55° C. for 60 min with occasional mixing by vortexing. The tubes were then incubated at 65° C. for 15 min with occasional vortexing. The higher temperature was employed to reverse formaldehyde crosslinking of the RNA. The tubes were then incubated at 80° C. for 15 min to deactivate the protease.

The sample was further processed by adding a 10× DNase I buffer (5 μL) and DNase I enzyme to each sample, which were then incubated for 15 min at RT. The solution was removed, and placed in a clean 1.5 mL tube and then processed with the Zymo Quick-RNA MicroPrep kit to clean the RNA from enzymes and elute the RNA into 154, of water. A reverse-transcription quantitative PCR (RT-qPCR) was conducted using the Luna Universal Probe One-step RT-qPCR kit (New England Biolabs, MA). A PCR reaction solution was made by adding forward and reverse primers (0.4 fluorescein (FAM)-labeled probe (0.2 μM) and BSA (1 mg/mL) to the PCR reaction solution and sealing. The selective amplification and corrected detection was conducted on a QuantStudio3 real-time PCR instrument (Applied Biosystems, CA) using Taqman chemistry, standard curve experiment, and cycle threshold analysis of 55° C. for 15 min, 95° C. for 1 min for 1 cycle, and then cycling at 10 sec at 95° C. followed by 60 sec at 60° C. for 1 min for up to 55 cycle, and finally storing the sample at 4° C. Positive and negative controls containing or lacking SKBR lysates were ran. The minimal cycle number was always less than 40 amplification cycles and used to determine if detection of a minimal copy number of CK19 mRNA of >about 10,000 was achieved (See Table 1).

Samples whether measured before or after antigen releasing achieved the minimal cycle number while maintaining a minimal antigen sensitivity and minimal nucleic acid number (See Table 1). The methods allowed release of minimal copy number and minimal cycle number. The samples were stable and the method was not destructive to captured nucleic acids. The method worked for both cell free and cellular samples. In contrast methods without the releasable analytical label were un-able to detect both the minimal antigen number and minimal nucleic acid number.

TABLE 1
Comparison of minimal antigen and nucleic acid detection

| | | | Minimal Antigen | Minimal |
|---|---|---|---|---|
| | Antigen/ | Antigen/ | sensitivity | Nucleic |
| | Nucleic | Nucleic | (50 cell | copy number |
| Case | Origin | Origin | equivalents) | >10,000 |
| 1 | After antigen | Cell free | Achieved | Achieved |
| | analysis | | | |
| 2 | After antigen | Cellular | Achieved | Achieved |
| | analysis | | | |
| 3 | Before antigen | Cell free | Achieved | Achieved |
| | analysis | | | |
| 4 | Before antigen | Cellular | Achieved | Achieved |
| | analysis | | | |

Commonly owned pending U.S. application Ser. No. 15/941,059 entitled Methods And Apparatus For Removal Of Small Volume From A Filtration Device filed Mar. 30, 2018 and Ser. No. 15/941,125 entitled Methods And Apparatus For Selective Nucleic Acid Analysis filed Mar. 30, 2018 are both incorporated by reference herein.

Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims. Furthermore, the foregoing description, for purposes of explanation, used specific nomenclature to provide a thorough understanding of the invention.

All patents, patent applications and publications cited in this application including all cited references in those patents, applications and publications, are hereby incorporated by reference in their entirety for all purposes to the same extent as if each individual patent, patent application or publication were so individually denoted.

While the many embodiments of the invention have been disclosed above and include presently preferred embodiments, many other embodiments and variations are possible within the scope of the present disclosure and in the appended claims that follow. Accordingly, the details of the preferred embodiments and examples provided are not to be construed as limiting. It is to be understood that the terms used herein are merely descriptive rather than limiting and that various changes, numerous equivalents may be made without departing from the spirit or scope of the claimed invention.

What is claimed is:

1. A method for the collection, purification and analysis of antigens and nucleic acids from the same sample, said method comprising:
(a) collecting and purifying said antigens and nucleic acids; and
(b) analyzing said antigens by means not destructive to the captured nucleic acids.

2. The method of claim 1, wherein said collected antigens and nucleic acids are retained in a cell, particle or droplet.

3. The method of claim 1, wherein said collected antigens and nucleic acids are retained on a porous matrix.

4. The method of claim 1, where undesired antigens and nucleic acids are washed way from retained antigens and nucleic acids.

5. The method of claim 1, wherein said antigens and nucleic acids are free of cells.

6. The method of claim 1, where said antigens and nucleic acids are cellular.

7. The method of claim 3, wherein said retained antigens and nucleic acids are sealed to protect them from contamination prior to analysis.

8. The method of claim 1, wherein said antigens are measured by releasing an analytical label, which is not destructive to the nucleic acids.

9. The method of claim 1, wherein said antigens are measured with an affinity agent and analytical label.

10. The method of claim 1, wherein said antigens are retained with an affinity agent.

11. The method of claim 1, wherein said nucleic acids are released from the sample after or before antigens are analyzed.

12. The method of claim 1, wherein said nucleic acids are amplified.

13. The method of claim 1, wherein said nucleic acids are released by lysis of cells.

14. The method of claim 1, wherein said antigen analyses are used to decide if nucleic acid amplification or measurement is warranted.

15. The method of claim 1, wherein said antigens and nucleic acids analyses are related to the health condition of a biological subject.


SEQUENCE LISTING (18 sequences)

SEQ ID NO: 1 - protein, 19 aa, Artificial Sequence
  Synthetic
MALWMRLLPLLALLALWGP

SEQ ID NO: 2 - protein, 10 aa, Artificial Sequence
  Synthetic
MALWMRLLPL

SEQ ID NO: 3 - protein, 9 aa, Artificial Sequence
  Synthetic
ALLALWGPD

SEQ ID NO: 4 - protein, 34 aa, Artificial Sequence
  Synthetic
AAAFVNQHLCGSHLVEALYLVCGERGFFYTPKTR

SEQ ID NO: 5 - protein, 23 aa, Artificial Sequence
  Synthetic
PAAAFVNQHLCGSHLVEALYLVC

SEQ ID NO: 6 - protein, 13 aa, Artificial Sequence
  Synthetic
PAAAFVNQHLCGS

SEQ ID NO: 7 - protein, 12 aa, Artificial Sequence
  Synthetic
CGSHLVEALYLV

SEQ ID NO: 8 - protein, 8 aa, Artificial Sequence
  Synthetic
VEALYLVC

SEQ ID NO: 9 - protein, 8 aa, Artificial Sequence
  Synthetic
LVCGERGF

SEQ ID NO: 10 - protein, 6 aa, Artificial Sequence
  Synthetic
FFYTPK

SEQ ID NO: 11 - protein, 31 aa, Artificial Sequence
  Synthetic
REAEDLQVGQVELGGGPGAGSLQPLALEGSL

SEQ ID NO: 12 - protein, 12 aa, Artificial Sequence
  Synthetic
REAEDLQVGQVE

SEQ ID NO: 13 - protein, 8 aa, Artificial Sequence
  Synthetic
LGGGPGAG

SEQ ID NO: 14 - protein, 11 aa, Artificial Sequence
  Synthetic
SLQPLALEGSL

SEQ ID NO: 15 - protein, 21 aa, Artificial Sequence
  Synthetic
GIVEQCCTSICSLYQLENYCN

SEQ ID NO: 16 - protein, 14 aa, Artificial Sequence
  Synthetic
GIVEQCCTSICSLY

SEQ ID NO: 17 - protein, 7 aa, Artificial Sequence
  Synthetic
QLENYCN

SEQ ID NO: 18 - protein, 7 aa, Artificial Sequence
  Synthetic
CSLYQLE